(19) United States
(12) Patent Application Publication
(10) Pub. No.: US 2008/0124279 A1
(43) Pub. Date: May 29, 2008

(54) Colonic delivery using Zn/pectin beads with a Eudragit coating

(76) Inventors: Antoine Andremont, Malakoff (FR); Helene Huguet, Paris (FR)

Correspondence Address: INTELLECTUAL PROPERTY / TECHNOLOGY LAW, PO BOX 14329, RESEARCH TRIANGLE PARK, NC, 27709, US

(21) Appl. No.: 11/985,465

(22) Filed: Nov. 15, 2007

Related U.S. Application Data
(60) Provisional application No. 60/859,600, filed on Nov. 17, 2006.

(51) Int. Cl. A61K 49/00; A61K 9/14; A61P 35/00; A61P 1/00; A61P 43/00

(52) U.S. Cl. 424/9.1; 424/493

(57) ABSTRACT

Drug delivery systems that can deliver therapeutic and/or diagnostic agents to the colon are disclosed. The systems include pectin beads crosslinked with zinc or any divalent cation of interest, which beads are then coated with Eudragit®-type polymers. The drug delivery systems are orally administrable, but can deliver the active agents to the colon. In some embodiments, they can administer the agents to various positions in the gastro-intestinal tract, including the colon. The agent can be a small molecule, peptide, protein, nucleic acid, or complex structures of natural, recombinant or synthetic origin. In still other embodiments, the agent is a diagnostic agent. The agents can be used to diagnose, treat or investigate humans and animals for a variety of conditions, including infectious diseases, inflammatory diseases, cancers and the like. Colon-specific delivery is obtained by formulating a prophylactic, therapeutic, and/or diagnostic agent with specific polymers that degrade in the colon, such as pectin. The pectin is gelled/crosslinked with a cation such as a zinc cation. The formulation, typically in the form of ionically crosslinked pectin beads, is subsequently coated with a specific polymer such as a Eudragit® polymer. Processes for obtaining such beads are also disclosed.

### REFERENCE TO RELATED APPLICATION

This application claims priority to U.S. Ser. No. 60/859,600, filed on Nov. 17, 2006, the contents of which are hereby incorporated by reference.

### FIELD OF THE INVENTION

The present invention is in the area of oral drug delivery systems to administer active agents to the colon.

### BACKGROUND OF THE INVENTION

Drug delivery systems that specifically deliver active agents to the colon have been recognized as having important therapeutic advantages. A large number of colonic conditions could effectively be treated more efficaciously if the active ingredient is released locally. Examples of such colonic disorders include Crohn's disease, ulcerative colitis, colorectal cancer and constipation.

Colonic release can also benefit patients when, from a therapeutic point of view, a delay in absorption is necessary. Examples include the treatment of disorders such as nocturnal asthma or angor (Kinget R. et al. (1998), Colonic Drug Targeting, Journal of Drug Targeting, 6. 129).

Colonic release can also be used to administer therapeutically active polypeptides. Polypeptides are typically administered by injection, because they are degraded in the stomach. Because injection is painful, research efforts have focused on using the colon as a site of absorption for active polypeptides, including analgesics, contraceptives, vaccines, insulin, and the like. The absorption of polypeptides in the colon appears to be more effective than in other sites in the digestive tract. This is particularly due to the relatively weak proteolytic activity in the small intestine and the absence of peptidase activity associated with the membrane of the colonic epithelial cells.

Following their oral administration, antibiotics pass through the stomach and are then absorbed in the small intestine to diffuse in the whole organism and treat the infectious outbreak site(s) for which they have been administered. All the same, a fraction of antibiotics ingested (the importance of this fraction varies with the characteristics of each antibiotic) is not absorbed and continues its progress to the colon before being eliminated in the stool. These residual antibiotics are combined, in the large intestine, with a fraction of the antibiotics absorbed, but which are re-excreted in the digestive tract by means of biliary elimination. This fraction is of variable importance as a function of metabolism and elimination pathways for each antibiotic. Finally, for certain antibiotics, a fraction of the dose absorbed is directly eliminated from the blood through the intestinal mucosa back into the lumen of the digestive tract, a good example is known with ciprofloxacin. Thus, whether administered orally or parenterally, a residual fraction of active antibiotics is generally found in the colon. This is the case, to varying degrees, for the great majority antibiotics from the various families used in therapeutics, with the sole notable exception of antibiotics from the amino-glycoside family for which intestinal excretion is negligible. For other antibiotics, intestinal excretion of a residual antibiotic activity will have a variety of consequences, all harmful. Indeed, the colon harbors a complex and very dense bacterial ecosystem (several hundreds of different bacterial species; more than 10¹¹ bacteria per gram of colonic content) which will be affected by the arrival of active antibiotic residues. The following can be observed:

1. Flora imbalance which is the main cause of banal diarrhea occurring following antibiotic treatments (Bartlett J. G. (2002) Clinical practice. Antibiotic associated diarrhea, New England Journal of Medicine, 346, 334). Even though this diarrhea is generally not serious and ceases rapidly, either spontaneously, or upon completion of the antibiotic treatment, it is adversely perceived by patients and adds to the discomfort of the original illness for which the antibiotic was prescribed;

2. interference with the resistance to colonization by exogenic bacteria (or “barrier effect”) with possible risk of infection, such as alimentary salmonella intoxication (Holmberg S. D. et al. (1984) Drug resistant Salmonella from animals fed antimicrobials, New England Journal of Medicine, 311, 617);

3. selection of microorganisms resistant to the antibiotic. These microorganisms can be of various types:

a) first they can be pathogenic bacteria such as for example, Clostridium difficile, a species capable of secreting toxins causing a form of colitis known as pseudomembranous colitis (Bartlett J. G. (1997) Clostridium difficle infection: pathophysiology and diagnosis, Seminar in Gastrointestinal Disease, 8, 12);
b) they can also be microorganisms that are relatively weakly pathogenic, but whose multiplication can lead to an associated infection (vaginal Candidosis or Escherichia coli resistant cystitis).

c) they can finally be non-pathogenic commensal drug-resistant bacteria whose multiplication and fecal elimination will increase dissemination of antibiotic resistance in the environment. It is well documented that antibiotic resistance genes are carried by mobile or transposable genetic elements that may contain up to 5 or 6 antibiotic resistance genes, and are readily transmitted to other bacteria, even across species. Consequently, these resistant commensal bacteria may constitute an important source leading to drug resistance for pathogenic species. This risk is currently considered seminal in terms of the disquieting character of the evolution towards drug multiresistance by numerous species pathogenic for humans.

It would therefore be desirable to have drugs and drug delivery systems that would act to reduce the quantity of residual antibiotics reaching the colon following oral or parenteral antibiotic therapy. To this end, it would be advantageous to have drug delivery systems which enable to administer the active agents to the colon.

Numerous strategies exploiting the diverse physiological parameters of the digestive tract have been devised with the aim to release active ingredients in the colon. These strategies have focused on drug delivery systems based on (1) using polymers that are sensitive to variations in pH, (2) time-dependent drug release form-s, (3) prodrugs or polymers degradable by bacteria of the intestinal flora.

The present invention provides drug delivery systems capable of delivering active agents to the colon.

### SUMMARY OF THE INVENTION

Drug delivery systems that can deliver prophylactic, therapeutic and/or diagnostic agents to the colon are disclosed. The systems include pectin beads crosslinked with zinc or any divalent cation of interest, which beads are then coated with Eudragit®-type polymers. The drug delivery systems are orally administrable, but can deliver the active agents to the colon. In some embodiments, they can administer the agents to various positions in the gastro-intestinal tract, including the colon.

In one embodiment, the therapeutic agent is a metallo-dependent enzyme. Application is illustrated for β-lactamase L1 from Stenotrophomonas maltophilia. However, one can also use β-lactamases which are not metallo-enzymes (classes A, C or D). Moreover, one can use enzymes, metallo-dependent or otherwise, to inactivate other classes of antibiotics such as macrolides, quinolones and fluoriquinolones, glycopeptides, lipopeptides, cyclins, oxazolidinones, and other classes of antibiotics. The enzymes can have the full sequence of the native enzyme, or can be truncated or otherwise modified so long as they maintain acceptable activity.

In other embodiments, the therapeutic agents include, but are not limited to:
peptides and proteins (including, but not limited to, enzymes, hormones, cytokines, lymphokines, growth factors, antibodies, and the like) whether natural, synthetic or recombinant;
nucleic acids and compounds including elements from nucleic acids (including, but not limited to, plasmids, oligonucleotides (oligoribonucleotides, deoxyribonucleotides, SiRNAs or ShRNAs of various lengths, and mixed molecules, including natural and/or modified bases, and optionally containing substitutions and modifications), as well as peptide nucleic acids;
complex structures of natural, recombinant or synthetic origin, including, but not limited to viruses (including DNA and RNA viruses, viruses targeting animal cells, viruses targeting vegetal cells, or viruses targeting bacteria better known as bacteriophages), bacteria (in whatever form, including spores), mycoplasms, yeasts and other unicellular eucaryotes (in whatever form, including spores)
natural, synthetic or mixed chemical molecules or mixtures thereof of any size, class or structure;
compounds for use in diagnosis, treatment or investigation of humans and animals for whatever reason or condition, including infectious diseases (including but not limited to those of bacterial and viral origin), inflammatory diseases, cancers,
compounds for assisting, complementing or modifying a treatment with anti-infectious agents, anti-inflammatory, anti-cancer agents, immuno-modifying agents, and the like, particularly where such assistance, complementation, or modification relates to the ability to block or modulate the activity of receptors in the colon, or inactivate other therapeutic agents which might modulate the activity of receptors in the colon.

Colon-specific delivery is obtained by formulating a prophylactic, therapeutic, and/or diagnostic agent with specific polymers that degrade in the colon, such as pectin. The pectin is gelled/crosslinked with a cation such as a zinc cation. The formulation, typically in the form of ionically crosslinked pectin beads, is subsequently coated with a specific polymer such as a Eudragit® polymer.

The delivery can be modulated to occur at various pre-selected sites of delivery within the intestinal tract by gelling/crosslinking a mixture of the prophylactic, therapeutic, and/or diagnostic agent and pectin, with divalent metallic cations such as Ca²⁺ or Zn²⁺.

Previous efforts have focused on coating pectin beads with cationic polymers such as polyethylene imine (PEI), chitosan or other cationic polymers, to prevent the pectin beads from degrading in the upper gastro-intestinal tract. Such efforts are described, for example, in U.S. patent application Ser. No. 10/524,318, and U.S. Patent Application No. 60/651,352, the contents of which are hereby incorporated by reference.

The present invention relates to coating the pectin beads with Eudragit® polymers such as FS30D, L30D (also known as L30D-55), NE30D, mixtures thereof or other desirable types of Eudragit® polymers to achieve the desired release of the prophylactic, therapeutic and/or diagnostic agent at predefined levels of the gastro-intestinal tract (GIT).

When the Eudragit® coating is dissolved, according to certain parameters such as pH or time, the beads are preferentially degraded by pectinolytic enzymes found in the lower part of the intestinal tract. Degradation of pectin then releases the prophylactic, therapeutic and/or diagnostic agent encapsulated within the bead.

One aspect of the invention is to provide a stable metallo-enzyme formulation for the lower intestinal or colonic delivery of such an enzyme. The use of zinc cations to crosslink the pectin is particularly preferred when specific metallo-dependent enzymes, which are Zn²⁺ dependent, could interact with other cationic species if they were used to gel the pectin beads. Such interactions could drastically affect the activity of such metallo-dependent enzymes. Accordingly, one embodiment of the drug delivery system involves using Zn²⁺ ions as a crosslinking agent for the pectin beads and in association with Zn²⁺ dependent enzymes which are very sensitive to the presence of other competitive cations. Of course, if the enzymes are dependent on other metal cations, such other metal cations (if they have a valence exceeding ⁺¹) can be used to crosslink the pectin.

The processes to obtain such beads can involve specific process conditions, such as time for gelification, washing, and drying that can be optimized to provide the highest quality beads, with optimized efficacy in vitro and in vivo. Therefore, another embodiment of the invention relates to processes for preparing zinc-crosslinked and Eudragit®-coated pectin beads.

### BRIEF DESCRIPTION OF THE FIGURES

FIG. 1 is a graph showing the efficiency of water rinsing to remove excess metallic cations from a formulation of β-Lacatamase L1 in pectin beads crosslinked with zinc acetate, measured in terms of conductivity (mS/cm) per sample following various washes.

FIG. 2 is a graph showing the effect of gelification time, rinsing process, and drying time on recovery of β-Lactamase L1 activity

FIG. 3 is a graph showing the enzymatic activity of β-lactamase L1 using CENTA as a substrate, measured in terms of response (OD/min) versus L1 concentration (μg/ml).

FIG. 4 is a series of scanning electron micrographs showing Eudragit-coated beads prepared using the methods described herein, and a cross-section of the beads showing the approximate thickness of Eudragit layer.

FIG. 5 is a chart showing the release kinetics of β-lactamase L1 from uncoated beads, and Eudragit-coated beads with or without hydroxypropyl methyl cellulose (HPMC) pre-coating, measured in terms of activity (μg/mg beads) versus time (minutes). Blue triangles represent uncoated beads; red circles represent beads coated with 40% Eudragit L30D-55 without pre-coating; green squares represent beads pre-coated with 5% HPMC and coated with 40% Eudragit L30D-55.

FIG. 6 is a chart showing the hydrolysis of amoxicillin by uncoated, and Eudragit-coated beads with or without a hydroxypropyl methylcellulose (HPMC) pre-coating, measured in terms of residual amoxicillin (%) versus time (minutes). Blue triangles represent uncoated beads; red circles represent beads coated with 40% Eudragit L30D-55 without pre-coating; green squares represent beads pre-coated with HPMC and coated with Eudragit L30D-55.

FIG. 7 is a chart showing the effect of Eudragit-coated pectin beads containing β-lactamase L1 on the emergence of antibiotic-resistant bacteria in piglets treated with amoxicillin, measured in terms of amoxicillin resistant bacteriacae (%) versus treatment duration (days). Blue triangles represent untreated animals (n=12); red diamonds represent animals treated with amoxicillin and placebo pectin beads (n=12); green squares represent animals treated with amoxicillin together with Eudragit-coated pectin beads containing β-lactamase L1 (n=4).

### DETAILED DESCRIPTION OF THE INVENTION

The drug delivery systems described herein will be better understood with reference to the following detailed description.

### I. Pectin Beads

The pectin beads are formed from pectin, zinc ions, and further coating with Eudragit® polymers and encapsulate one or more active agents.

Stability and protection of the pectin beads in gastric medium and intestinal medium is ensured by the Eudragit® polymer coating. In contrast, uncoated beads of pectin tend not be stable in such an environment and may not adequately protect their contents against degradation and/or inactivation. The Eudragit® coating ensures that they resist long enough so that their contents able to reach the colon intact.

Pectin

Pectin is it polysaccharide isolated from the cellular walls of superior plants, used widely in the agricultural food industry (as a coagulant or thickener for jams, ice creams and the like) and pharmaceutics. It is polymolecular and polydisperse. Its drug delivery system varies depending on the source, extraction conditions and environmental factors.

Pectins are principally composed of linear chains of beta-1,4-(D)-galacturonic acid, at times interspersed by units of rhamnose. The carboxylic groups of galacturonic acid can be partially esterified to yield methylated pectins. Two types of pectins are distinguished according to their degree of methylation (DM: number of methoxy groups per 100 units of galacturonic acid):
highly methylated pectin (HM: high methoxy) where the degree of methylation varies between 50 and 80%. It is slightly soluble in water and forms gels in acidic medium (pH<3.6) or in the presence of sugars;
weakly methylated pectin (LM: low methoxy), with a degree of methylation varying from 25 to 50%. More soluble in water than HM pectin, it gives gels in the presence of divalent cations such as Ca²⁺ ions. Indeed, Ca²⁺ ions form “bridges” between the free carboxylated groups of galacturonic acid moities. The network that is formed has been described by Grant et al. under the name of <<egg-box model>> (Grant G. T. et al. (1973) Biological interactions between polysaccharides and divalent cations: the egg-box model, FEBS Letters, 32, 195).

There are also amidated pectins. Treatment of pectin by ammonia transforms some methyl carboxylate groups (—COOCH₃) into carboxamide groups (—CONH₂). This amidation confers novel properties to the pectins, in particular better resistance to variations in pH. Amidated pectins tend to be more tolerant to the variations in pH, and have also been studied for the manufacture of matricial tablets for colonic delivery (Wakerly Z. et al. (1997) Studies on amidated pectins as potential carriers in colonic drug delivery, Journal of Pharmacy and Pharmacology. 49, 622).

Pectin is degraded by enzymes originating from higher plants and various microorganisms (fungi, bacteria, and the like) among which bacteria from the human colonic flora. The enzymes produced by the microflora encompass a mixture of polysaccharidases, glycosidases and esterases.

Zinc Cations

Divalent zinc cations from various zinc salts can be used to crosslink pectin. Examples include zinc sulfate, zinc chloride, and zinc acetate.

Eudragit® Polymers

The coating of drug-loaded cores such as tablets, capsules, granules, pellets or crystals offers many advantages over uncoated counterparts, such as higher physicochemical stability, better compliance and increased therapeutic efficiency of the active ingredients. Indeed, the effectiveness of a medication depends not only on the actives it contains, but also on formulation and processing.

Poly(meth)acrylates have proven particularly suitable as coating materials. These polymers, typically used in amounts of only a few milligrams, are pharmacologically inactive, i.e. are excreted unchanged.

EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph.Eur., USP/NF, DMF and JPE.

EUDRAGIT® polymers can provide the following possibilities for controlled drug release:
Gastrointestinal tract targeting (gastroresistance, release in the colon)
Protective coatings (taste and odor masking, protection against moisture)
Delayed drug release (sustained-release formulations).

### EUDRAGIT® polymers are available in a wide range of different concentrations and physical forms, including aqueous solutions, aqueous dispersion, organic solutions, and solid substances.

The pharmaceutical properties of EUDRAGIT® polymers are determined by the chemical properties of their functional groups. A distinction is made between:
poly(meth)acrylates, soluble in digestive fluids (by salt formation)

EUDRAGIT® L, S, FS and E polymers with acidic or alkaline groups enable pH-dependent release of the active ingredient.

Applications: from simple taste masking via resistance solely to gastric fluid, to controlled drug release in) all sections of the intestine.
poly(meth)acrylates, insoluble in digestive fluids

EUDRAGIT® RL and RS polymers with alkaline and EUDRAGIT® NE polymers with neutral groups enable controlled time release of the active by pH-independent swelling.

Enteric Coatings: Gastoresistance and Release in the Colon

Enteric EUDRAGIT® coatings provide protection against drug release in the stomach and enable controlled release in the intestine. Targeted drug release in the gastrointestinal tract is recommended for particular applications or therapeutic strategies, for example when the drug is sparingly soluble in the upper digestive tract, or when the drug may be degraded by gastric fluid. Secondly, this dosage form is very patient-friendly as it does not stress the stomach and the number of doses of the therapeutic drug can be considerably reduced, thanks to prolonged delivery. The dominant criterion for release is the pH-dependent dissolution of the coating, which takes place in a certain section of the intestine (pH 5 to over 7) rather than in the stomach (pH 1-5). For these applications, anionic EUDRAGIT® grades containing carboxyl groups can be mixed with each other. This makes it possible to finely adjust the dissolution pH, and thus to define the drug release site in the intestine. EUDRAGIT® L and S grades are suitable for enteric coatings. EUDRAGIT® FS 30 D is specifically used for controlled release in the colon.

Application benefits of enteric EUDRAGIT® coatings include:
pH-dependent drug release
protection of actives sensitive to gastric fluid
protection of the gastric mucosa from aggressive actives
increase in drug effectiveness
good storage stability
controlled release in the colon/GI targeting

Active Agents

The active agent can be an anti-infectious, for example antibiotics, anti-inflammatory compounds, anti-histamines, anti-cholinergics, antivirals, antimitotics, peptides, proteins, enzymes, nucleic acids (RNA or DNA), peptide nucleic acids, plasmids, genes, anti-sense oligonucleotides, interfering RNAs, ribozymes, small molecules with specific binding capacities or activities (such as targeted chemotherapeutics), diagnostic agents, immunosuppressive agents, viruses, bacteria, other micro-organisms or eukaryotic cells.

The active agent can be introduced into the drug delivery system as a powder, a solution, a suspension, or complexed with a solubilizing agent, such as a cyclodextrin or any other suitable compound.

Some of the active agents described herein can be administered in the form of prodrugs. Prodrugs have been widely studied for the colonic targeting of various active ingredients (such as steroid and non-steroid anti-inflammatory drugs, and spasmolytics). These systems are based on the capacity of the enzymes produced by the colonic flora to act on the prodrugs to release the active form of the active ingredient.

The prodrugs can be based on the action of bacterial azoreductases, so that the active agents are targeted to the colon with the drug delivery systems described herein, and the active agents are formed by reaction of the prodrug with a bacterial azoreductase, which provides a dual mechanism for ensuring that the drugs are administered to the colon. Representative chemistry for forming such prodrugs is described, for example, in Peppercorn M. A. et al. (1972) The role of intestinal bacteria in the metabolism of salicylazosulfapyridin, The Journal of Pharmacology and Experimental Therapeutics., 181, 555 and 64, 240.

Another approach consists in using bacterial hydrolases such as glycosidases and polysaccharidases (Friend D. R. (1995) Glycoside prodrugs: novel pharmacotherapy for colonic diseases, S.T.P. Pharma Sciences, 5, 70; Friend D. R. et al. (1984) A colon-specific drug-delivery system based on drug glycosides and the glycosidases of colonic bacteria, Journal of Medicinal Chemistry, 27, 261; Friend D. R. et al. (1985) Drug glycosides: potential prodrugs for colon-specific drug delivery, Journal of Medicinal Chemistry. 28, 51; and Friend D. R. et al. (1992) Drug glycosides in oral colon-specific drug delivery, Journal of Controlled Release, 19, 109). Prodrugs have thus been developed by coupling, for example, sugar with steroids (glucose, galactose, cellobiose, dextrane (international application WO 90/09168)), cyclodextrins Hirayama F. et al. (1996) hi vitro evaluation of Biphenylyl Acetic Acid-beta-Cyclodextrin conjugates as colon-targeting prodrugs: drug release behavior in rat biological media, Journal of Pharmacy and Pharmacology, 49, 27).

a) Agents that Inactivate Antibiotics

In one embodiment, the active agent is an enzyme capable of inactivating antibiotics in the colon. When the antibiotic is a beta-lactam antibiotic, β-lactamases can be used. The selected enzyme, i.e. β-lactamase L1, a Zn²⁺-dependent β-lactamase from Stenotrophomonas maltophilia, was chosen from a series of β-lactamases because its characteristics showed the best profile for the targeted application. Also, it has been demonstrated to have an excellent stability profile. The characteristics of various β-lactamases evaluated are described hereafter.

TABLE 1

| | FEZ-1 * | | K_{cat}/Kₘ | L-1(wt) ** | | K_{cat}/Kₘ |
|---|---|---|---|---|---|---|
| Antibiotics | K_{cat} (s⁻¹) | Kₘ (μM) | (μM/s⁻¹) | K_{cat} (s⁻¹) | Kₘ (μM) | (μM/s⁻¹) |
| Penicillin | | | | | | |
| Benzylpenicillin | 70 | 590 | 0.11 | 600 | 38 | 16 |
| Ampicillin | >5.5 | >5000 | 0.011 | 520 | 55 | 9.5 |
| Cabenicillin | 35 | 1600 | 0.023 | | | |
| Pipericillin | 50 | 4200 | 0.012 | | | |
| Azlocillin | | | | | | |
| Mezlocillin | | | | | | |
| Ticarcillin | >65 | >5000 | 0.013 | | | |
| Timocillin | | | | | | |
| Cephalosporin | | | | | | |
| Cephaloridin | 16 | 1000 | 0.016 | | | |
| Cephalothin | 300 | 120 | 2.5 | 82 | 8.9 | 9.2 |
| Cefoxitin | | | | 1 | 3 | 0.33 |
| Cefuroxin | | | | | | |
| Cefotaxim | 165 | 70 | 2.4 | 270 | 10 | 27 |
| Ceftazidin | | | | | | |
| Cefepim | >6 | >1000 | 0.006 | | | |
| Cefpirom | | | | | | |
| Nitrocefin | 90 | 100 | 0.9 | 41 | 4 | 10 |
| Moxalactam | 3 | 18 | 0.17 | | | |
| Carbapenem | | | | | | |
| Imipenem | >200 | >1000 | 0.2 | 370 | 57 | 6.5 |
| Meropenem | 45 | 85 | 0.5 | 157 | 15 | 10 |
| Biapenem | 70 | >1000 | 0.07 | 134 | 32 | 4.2 |
| Monobactams | | | | | | |
| Aztreonam | | | | | | |
| Carumonam | | | | | | |
| Mechanism-Based | | | | | | |
| Inactivators | | | | | | |
| Sulbactam | | | | | | |
| Tazobactam | 40 | 700 | 1.06 | | | |
| Clavulanic Acid | <0.01 | >1000 | <0.00001 | 11 | 22 | 0.5 |

| | IMP-1 *** | | K_{cat}/Kₘ | VIM-2 **** | | K_{cat}/Kₘ |
|---|---|---|---|---|---|---|
| Antibiotics | K_{cat} (s⁻¹) | Kₘ (μM) | (μM/s⁻¹) | K_{cat} (s⁻¹) | Kₘ (μM) | (μM/s⁻¹) |
| Penicillin | | | | | | |
| Benzylpenicillin | 320 | 520 | 0.62 | 56 | 49 | 1.114 |
| Ampicillin | 950 | 200 | 4.8 | 125 | 90 | 1.4 |
| Cabenicillin | | | 0.02 | 185 | 205 | 0.9 |
| Pipericillin | | | 0.72 | 300 | 125 | 2.4 |
| Azlocillin | | | | 200 | 200 | 1 |
| Mezlocillin | | | | 200 | 125 | 1.4 |
| Ticarcillin | 1.1 | 740 | 0.0015 | 180 | 125 | 1.6 |
| Timocillin | | >2000 | <0.0001 | 7.7 | 390 | 0.002 |
| Cephalosporin | | | | | | |
| Cephaloridin | 53 | 22 | 2.4 | 140 | 50 | 2.8 |
| Cephalothin | 48 | 21 | 2.4 | 130 | 11 | 12 |
| Cefoxitin | 16 | 8 | 2 | 15 | 13 | 1.2 |
| Cefuroxin | 8 | 37 | 0.22 | 8 | 20 | 0.4 |
| Cefotaxim | 1.3 | 4 | 0.45 | 70 | 12 | 5.8 |
| Ceftazidin | 8 | 44 | 0.18 | 3.6 | 72 | 0.05 |
| Cefepim | 7 | 11 | 0.66 | >40 | >400 | 0.1 |
| Cefpirom | 9 | 14 | 0.64 | 180 | 180 | 1 |
| Nitrocefin | 63 | 27 | 2.3 | 770 | 18 | 43 |
| Moxalactam | | | | 90 | 55 | 1.6 |
| Carbapenem | | | | | | |
| Imipenem | 46 | 39 | 1.2 | 34 | 9 | 3.8 |
| Meropenem | 50 | 10 | | 5 | 2 | 2.5 |
| Biapenem | 160 | 28 | 6 | 8.5 | 15 | 0.55 |
| Monobactams | | | | | | |
| Aztreonam | >0.01 | >1000 | <0.0001 | <0.01 | >1000 | <0.0001 |
| Carumonam | >0.01 | >1000 | <0.0001 | | | |
| Mechanism-Based | | | | | | |
| Inactivators | | | | | | |
| Sulbactam | | | | 23 | 320 | 0.072 |
| Tazobactam | | | | 28 | 875 | 0.032 |
| Clavulanic Acid | | | | | | |

* (Mercuri et al., Antimicrob. Agents. Chemother. 2001 Apr; 45(4): 1254-1262)
** (Carenbauer et al., BMC Biochem. 2002; 3: 4. Epub 2002 Feb. 13; Frere, 2005, unpublished data)
*** (Murphy et al., 2003, Antimicrob. Ag. Chemother. 2003 Feb, 47(2): 582-7; Laraki et al., Antimicrob. Ag. Chemother. 1999 Apr., 43(4): 902-6)
**** (Docquier et al., J. Antimicrob. Chemother. 2003 Feb., 51(2): 257-266)

To inactivate antibiotics from other classes than beta-lactams, appropriate enzymes can be used. For example, one can use an erythromycin esterase to inactivate macrolide antibiotics.

b) Agents that Treat Colon Cancer

When the drug delivery systems are used to treat colon cancer, any type of antitumor agent can be used. The anti-tumor agents can be, for example, anti-proliferative agents, agents for DNA modification or repair, DNA synthesis inhibitors, DNA/RNA transcription regulators, RNA processing inhibitors, agents that affect protein expression, synthesis and stability, agents that affect protein localization or their ability to exert their physiological action, agents that interfere with protein-protein or protein-nucleic acid interactions, agents that act by RNA interference, receptor binding molecules of any chemical nature (including small molecules and antibodies), targeted toxins, enzyme activators, enzyme inhibitors, gene regulators, HSP-90 inhibitors, molecules interfering with microtubules or other cytoskeletal components or cell adhesion and motility, agents for phototherapy, and therapy adjuncts.

Representative antiproliferative agents include N-acetyl-D-sphingosine (C₂ ceramide), apigenin, berberine chloride, dichloromethylenediphosphonic acid disodium salt, loe-emodine, emodin, HA 14-1, N-hexanoyl-D-sphingosine (C₆ ceramide), 7b-hydroxycholesterol, 25-hydroxycholesterol, hyperforin, parthenolide, and rapamycin.

Representative agents for DNA modification and repair include aphidicolin, bleomycin sulfate, carboplatin, carmustine, chlorambucil, cyclophosphamide monohydrate, cyclophosphamide monohydrate ISOPAC®, cis-diammineplatinum(II) dichloride (Cisplatin), esculetin, melphalan, methoxyamine hydrochloride, mitomycin C, mitoxantrone dihydrochloride, oxaliplatin, and streptozocin.

Representative DNA synthesis inhibitors include (±)amethopterin (methotrexate), 3-amino-1,2,4-benzotriazine 1,4-dioxide, aminopterin, cytosine b-D-arabinofurdnoside (Ara-C), cytosine b-D-arabinofuranoside (Ara-C) hydrochloride, 2-fluoroadenine-9-b-D-arabinofuranoside (Fludarabine des-phosphate; F-ara-A), 5-fluoro-5′-deoxyuridinc, 5-fluorouracil, ganciclovir, hydroxyurea, 6-mercaptopurine, and 6-thioguanine.

Representative DNA/RNA transcription regulators include actinomycin D, daunorubicin hydrochloride, 5,6-dichlorobenzimidazole 1-b-D-ribofuranoside, doxorubicin hydrochloride, homoharringtonine, and idarubicin hydrochloride.

Representative enzyme activators and inhibitors include forskolin, DL-aminoglutethimide, apicidin, Bowman-Birk Inhibitor, butein, (S)-(+)-camptothecin, curcumin, (−)-deguelin, (−)-depudecin, doxycycline hyclate, etoposide, formestane, fostriecin sodium salt, hispidin, 2-imino-1-imidazolidineacetic acid (Cyclocreatine), oxamflatin, 4-phenylbutyric acid, roscovitine, sodium valproate, trichostatin A, tyrphostin AG 34, tyrphostin AG 879, urinary trypsin inhibitor fragment, valproic acid (2-propylpentanoic acid), and XK469.

Representative gene regulators include 5-aza-2′-deoxycytidine, 5-azacytidine, cholecalciferol (Vitamin D3), ciglitizone, cyproterone acetate, 15-deoxy-D^{12,14}-prostaglandin J₂, epitestosterone, flutamide, glycyrrhizic acid ammonium salt (glycyrrhizin), 4-hydroxytamoxifen, mifepristone, procainamide hydrochloride, raloxifene hydrochloride, all trans-retinal (vitamin A aldehyde), retinoic acid (vitamin A acid), 9-cis-retinoic acid, 13-cis-retinoic acid, retinoic acid p-hydroxyanilide, retinol (Vitamin A), tamoxifen, tamoxifen citrate salt, tetradecylthioacetic acid, and troglitazone.

Representative HSP-90 inhibitors include 17-(allylamino)-17-demethoxygeldanamycin and geldanamycin.

Representative microtubule inhibitors include colchicines, dolastatin 15, nocodazole, taxanes and in particular paclitaxel, podophyllotoxin, rhizoxin, vinblastine sulfate salt, vincristine sulfate salt, and vindesine sulfate salt and vinorelbine (Navelbine) ditartrate salt.

Representative agents for performing phototherapy include photoactive porphyrin rings, hypericin, 5-methoxypsoralen, 8-methoxypsoralen, psoralen and ursodeoxycholic acid.

Representative agents used as therapy adjuncts include amifostine, 4-amino-1,8-naphthalimide, brefeldin A, cimetidine, phosphomycin disodium salt, leuprolide (leuprorelin) acetate salt, luteinizing hormone-releasing hormone (LH-RH) acetate salt, lectin, papaverine hydrochloride, pifithrin-a, (−)-scopolamine hydrobromide, and thapsigargin.

The agents can also be anti-VEGF (vascular endothelial growth factor) agents, as such are known in the art. Several antibodies and small molecules are currently in clinical trials or have been approved that function by inhibiting VEGF, such as Avastin (Bevacizumab), SU5416, SU11248 and BAY 43-9006. The agents can also be directed against growth factor receptors such as those of the EGF/Erb-B family such as EGF Receptor (Iressa or Gefitinib, and Tarceva or Erlotinib), Erb-B2, receptor (Herceptin or Trastuzumab), other receptors (such as Rituximab or Rituxan/MabThera), tyrosine kinases, non-receptor tyrosine kinases, cellular serine/threonine kinases (including MAP kinases), and various other proteins whose deregulation contribute to oncogenesis (such as small/Ras family and large/heterotrimeric G proteins). Several antibodies and small molecules targeting those molecules are currently at various stages of development (including approved for treatment or in clinical trials).

Some of the most commonly used antitumor agents currently in use or in clinical trials include paclitaxel, docetaxel, tamoxifen, vinorelbine, gemcitabine, cisplatin, etoposide, topotecan, irinotecan, anastrozole, rituximab, trastuzumab, fludarabine, cyclophosphamide, gentuzumab, carboplatin, interferons, and doxorubicin. The most commonly used anticancer agent is paclitaxel, which is used alone or in combination with other chemotherapy drugs such as: 5-FU, doxorubicin, vinorelbine, cytoxan, and cisplatin.

Combination therapy can be provided by combining two or more of the above compounds.

c) Agents that Treat Chrohn's Disease

There are several therapeutic approaches for treating Chrohn's Disease. Most people are first treated with drugs containing mesalamine, a substance that helps control inflammation. Sulfasalazine is the most commonly used of these drugs. Patients who do not benefit from it or who cannot tolerate it may be put oil other mesalamine-containing drugs, generally known as 5-ASA agents, such as Asacol, Dipentum, or Pentasa. Corticosteroids are often administered to control inflammation.

Immunosuppressive agents are also used to treat Crohn's disease. Most commonly prescribed are 6-mercaptopurine and a related drug, azathioprine. Immunosuppressive agents work by blocking the immune reaction that contributes to inflammation.

Patients can be treated with combinations of these agents, for example, combinations of corticosteroids and immunosuppressive drugs.

The U.S. Food and Drug Administration has approved the drug infliximab (brand name, Remicade) for the treatment of moderate to severe Crohn's disease that does not respond to standard therapies (mesalamine substances, corticosteroids, immunosuppressive agents) and for the treatment of open, draining fistulas. Infliximab is an anti-tumor necrosis factor alpha (TNF-alpha) antibody. This and other anti-TNF-alpha agents can be used to remove TNF-alpha from the colon, thereby preventing inflammation, without the side effects that might result if TNF-alpha were removed from the blood stream outside of the colon.

Antidiarrheal agents are often also administered, including diphenoxylate, loperamide, and codeine.

d) Agents that Treat Ulcerative Colitis

The agents that are used to treat ulcerative colitis overlap with those used to treat Chrohn's Disease. Examples include aminosalicylates, drugs that contain 5-aminosalicyclic acid (5-ASA), to help control inflammation, such as sulfasalazine, olsalazine, mesalamine, and balsalazide. They also include corticosteroids such as prednisone and hydrocortisone, and immunomodulators such as azathioprine and 6-mercapto-purine (6-MP), cytokines, interleukins, and lymphokines. Cyclosporine A may be used with 6-MP or azathioprine to treat active, severe ulcerative colitis. Anti-TNF-alpha agents, the thiazolidinediones or glitazones, including rosiglitazone and pioglitazone, can also be used.

c) Agents that Treat Constipation/Irritable Bowel Syndrome

Constipation, such as that associated with irritable bowel syndrome, is often treated using stimulant laxatives, osmotic laxatives such as Lactulose and MiraLax, stool softeners (such as mineral oil or Colace), bulking agents (such as Metamucil or bran). Agents such as Zelnorm (also called tegaserod) can be used to treat IBS with constipation. Additionally, anticholinergic medications such as Bentyl® and Levsin® have been found to be helpful in alleviating the bowel spasms of IBS.

f) Protein and Peptide Drugs

The drug delivery systems can be used to orally administer proteins and peptides that might otherwise be degraded if orally administered, and which might otherwise have to be administered intramuscularly or intravenously.

Examples of protein and peptide drugs useful in the present invention include:

Adrenocorticotropic hormone (ACTH) peptides including, but not limited to, ACTH, human; ACTH 1-10; ACTH 1-13, human; ACTH 1-16, human; ACTH 1-17; ACTH 1-24, human; ACTH 4-10; ACTH 4-11; ACTH 6-24; ACTH 7-38, human; ACTH 18-39, human; ACTH 4, rat; ACTH 12-39, rat; beta-cell tropin (ACTH 22-39); biotinyl-ACTH 1-24, human; biotinyl-ACTH 7-38, human; corticostatin, human; corticostatin, rabbit; [Met(02)⁴, DLys⁸, Phe⁹] ACTH 4-9, human; [Met(0)⁴, DLys⁸, Phe⁹] ACTH 4-9, human; N-acetyl, ACTH 1-17, human; and ebiratide.

Adrenomedullin peptides including, but not limited to, adrenomedullin, adrenomedullin 1-52, human; adrenomedullin 1-12, human; adrenomedullin 13-52, human; adrenomedullin 22-52, human; pro-adrenomedullin 45-92, human; pro-adrenomedullin 153-185, human; adrenomedullin 1-52, porcine; pro-adrenomedullin (N-20), porcine; adrenomedullin 1-50, rat; adrenomedullin 11-50, rat; and proAM-N20 (proadrenomedullin N-terminal 20 peptide), rat.

Allatostatin peptides including, but not limited to, allatostatin I; allatostatin II; allatostatin III; and allatostatin IV.

Amylin peptides including, but not limited to, acetyl-amylin 8-37, human; acetylated amylin 8-37, rat; AC187 amylin antagonist; AC253 amylin antagonist; AC625 amylin antagonist; amylin 8-37, human; amylin (IAPP), cat, amylin (insulinoma or islet amyloid polypeptide (IAPP)); amylin amide, human; amylin 1-13 (diabetes-associated peptide 1-13), human; amylin 20-29 (IAPP 20-29), human; AC625 amylin antagonist; amylin 8-37, human; amylin (IAPP), cat; amylin, rat; amylin 8-37, rat; biotinyl-amylin, rat; and biotinyl-amylin amide, human.

Amyloid beta-protein fragment peptides including, but not limited to, Alzheimer's disease beta-protein 12-28 (SP17); amyloid beta-protein 25-35; amyloid beta/A4-protein precursor 328-332; amyloid beta/A4 protein precursor (APP) 319-335; amyloid beta protein 1-43; amyloid beta-protein 1-42; amyloid beta-protein 1-40; amyloid beta-protein 10-20; amyloid beta-protein 22-35; Alzheimer's disease beta-protein (SP28); beta-amyloid peptide 1-42, rat: beta-amyloid peptide 1-40, rat; beta-amyloid 1-11; beta-amyloid 31-35; beta-amyloid 32-35; beta-amyloid 35-25; beta-amyloid/A4 protein precursor 96-110; beta-amyloid precursor protein 657-676; beta-amyloid 1-38; [Gln¹¹]-Alzheimer's disease beta-protein; [Gln¹¹]-beta-amyloid 1-40; [Gln²²]-beta-amyloid 6-40; non-A beta component of Alzheimer's disease amyloid (NAC); P3, (A beta 17-40) Alzheimer's disease amyloid β-peptide; and SAP (serum amyloid P component) 194-204.

Angiotensin peptides including, but not limited to, A-779; Ala-Pro-Gly-angiotensin II; [Ile³, Val⁵]-angiotensin II; angiotensin III antipeptide; angiogenin fragment 108-122; angiogenin fragment 108-123; angiotensin I converting enzyme inhibitor; angiotensin I, human; angiotensin I converting enzyme substrate; angiotensin 11-7, human; angiopeptin; angiotensin II, human; angiotensin II antipeptide; angiotensin II 1-4, human; angiotensin II 3-8, human; angiotensin II 4-8, human; angiotensin II 5-8, human; angiotensin III ([Des-Asp¹]-angiotensin II), human; angiotensin III inhibitor ([Ile⁷]-angiotensin III); angiotensin-converting enzyme inhibitor (Neothunnus macropterus); [Asn¹, Val⁵]-angiotensin I, goosefish; [Asn¹, Val⁵, Asn⁹]-angiotensin I, salmon; [Asn¹, Val⁵, Gly⁹]-angiotensin I, eel; [Asn¹, Val⁵]-angiotensin I 1-7, eel, goosefish, salmon; [Asn¹, Val⁵]-angiotensin II; biotinyl-angiotensin I, human; biotinyl-angiotensin II, human; biotinyl-Ala-Ala-Ala-angiotensin II; [Des-Asp¹]-angiotensin I, human; [p-aminophenylalanine⁶]-angiotensin II, renin substrate (angiotensinogen 1-13), human; preangiotensinogen 1-14 (renin substrate tetradecapeptide), human; renin substrate tetradecapeptide (angiotensinogen 1-14), porcine; [Sar¹]-angiotensin II, [Sar¹]-angiotensin II 1-7 amide; [Sar¹, Ala⁸]-angiotensin II; [Sar¹, Ile⁸]-angiotensin II; [Sar¹, Thr⁸]-angiotensin II; [Sar¹, Tyr(Me)⁴]-angiotensin II (Sarmesin); [Sar¹, Val⁵, Ala⁸]-angiotensin II; [Sar¹, Ile⁷]-angiotensin III; synthetic tetradecapeptide renin substrate (No. 2); [Val⁴]-angiotensin III; [Val⁵]-angiotensin II; [Val⁵]-angiotensin I, human; [Val⁵]-angiotensin I; [Val⁵, Asn⁹]-angiotensin I, bullfrog; and [Val⁵, Ser⁹]-angiotensin I, fowl.

Antibiotic peptides including, but not limited to, Ac-SQNY; bactenecin, bovine; CAP 37 (20-44); carbormethoxycarbonyl-DPro-DPhe-OBzl; CD36 peptide P 139-155; CD36 peptide P 93-110; cecropin A-melittin hybrid peptide [CA(1-7)M(2-9)NH2]; cecropin B, free acid; CYS(Bzl)84 CD fragment 81-92; defensin (human) HNP-2; dermaseptin; immunostimulating peptide, human; lactoferricin, bovine (BLFC); and magainin spacer.

Antigenic polypeptides, which can elicit an enhanced immune response, enhance an immune response and or cause an immunizingly effective response to diseases and/or disease causing agents including, but not limited to, adenoviruses; anthrax; Bordetella pertussus; botulism; bovine rhinotracheitis; Branhamella catarrhalis; canine hepatitis; canine distemper; Chlamydiae; cholera; coccidiomycosis: cowpox; cytomegalovirus; Dengue fever; dengue toxoplasmosis; diphtheria; encephalitis; enterotoxigenic Escherichia coli; Epstein Barr virus; equine encephalitis; equine infectious anemia; equine influenza; equine pneumonia; equine rhinovirus; Escherichia coli; feline leukemia; flavivirus; globulin; haemophilus influenza type b; Haemophihus influenzae; Haemophilus pertussis; Helicobacter pylor; hemophilus ??; hepatitis ??; hepatitis virus A; hepatitis virus B; Hepatitis virus C; herpes viruses; HIV; HIV-1 viruses; HIV-2 viruses; HTLV I; HTLV II; HTLV III; influenza ?; Japanese encephalitis; Klebsiellae species; Legionella pneumophila; leishmania; leprosy; lyme disease; malaria immunogen; measles; meningitis; Meningococcus; Meningococcal polysaccharide group A; Meningococcal polysaccharide group C; mumps; mumps virus; mycobacteria; Mycobacterium tuberculosis; Neisseria; Neisseria gonorrhoeae; Neisseria meningitidis; ovine blue tongue; ovine encephalitis; papilloma viruses; parainfluenza; paramyxoviruses; Pertussis toxins; plague; pneumococcus; Pneumocystis carinii; pneumonia; poliovirus; Proteus species; Pseudomonas aeruginosa; rabies; respiratory syncytial virus; rotavirus; rubella; Salmonellae; schistosomiasis; shigellae; simian immunodeficiency virus; smallpox; Staphylococcus aureus; Staphylococcus species; Streptococcus pneumoniae; Streptococcus pyogenes; Streptococcus species; Clostridium difficile; Clostridium species; swine influenza; tetanus; Treponema pallidum; typhoid; vaccinia; varicella-zoster virus; and vibrio cholerae.

Anti-microbial peptides including, but not limited to, buforin I; buforin II; cecropin A; cecropin B; cecropin P1, porcine; gaegurin 2 (Rana rugosa); gaegurin 5 (Rana rugosa); indolicidin; protegrin-(PG)-I; magainin 1; and magainin 2; and T-22 [Tyr^{5,12}, Lys⁷]-poly-phemusin II peptide.

Apoptosis related peptides including, but not limited to, Alzheimer's disease beta-protein (SP28); calpain inhibitor peptide; capsase-1 inhibitor V; capsase-3, substrate IV; caspase-1 inhibitor I, cell-permeable: caspase-1 inhibitor VI; caspase-3 substrate III, fluorogenic; caspase-1 substrate V, fluorogenic; caspase-3 inhibitor I, cell-permeable; caspase-6 ICE inhibitor III; [Des-Ac, biotin]-ICE inhibitor III; IL-1 B converting enzyme (ICE) inhibitor II; IL-1 B converting enzyme (ICE) substrate IV; MDL 28170; and MG-132.

Atrial natriuretic peptides including, but not limited to, alpha-ANP (alpha-chANP), chicken; anantin; ANP 1-11, rat; ANP 8-30, frog; ANP 11-30, frog; ANP-21 (fANP-21), frog; ANP-24 (fANP-24), frog; ANP-30, frog; ANP fragment 5-28, human, canine; ANP-7-23, human; ANP fragment 7-28, human, canine; alpha-atrial natriuretic polypeptide 1-28, human, canine; A71915, rat; atrial natriuretic factor 8-33, rat; atrial natriuretic polypeptide 3-28, human; atrial natriuretic polypeptide 4-28, human, canine; atrial natriuretic polypeptide 5-27; human; atrial natriuretic aeptide (ANP), eel; atriopeptin I, rat, rabbit, mouse; atriopeptin II, rat, rabbit, mouse; atriopeptin III, rat, rabbit, mouse; atrial natriuretic factor (rANF), rat, auriculin A (rat ANF 126-149); auriculin B (rat ANF 126-150); beta-ANP (1-28, dimer, antiparallel); beta-rANF 17-48; biotinyl-alpha-ANP 1-28, human, canine; biotinyl-atrial natriuretic factor (biotinyl-rANF), rat; cardiodilatin 1-16, human; C-ANF 4-23, rat; Des-[Cys¹⁰⁵, Cys¹²¹]-atrial natriuretic factor 104-126, rat; [Met(O)¹²] ANP 1-28, human; [Mpr⁷, DAla⁹]ANP 7-29, amide, rat; prepro-ANF 104-116, human; prepro-ANF 26-55 (proANF 1-30), human; prepro-ANF 56-92 (proANF 31-67), human; prepro-ANF 104-123, human; [Tyr⁰]-atriopeptin I, rat, rabbit, mouse; [Tyr⁰]-atriopeptin II, rat, rabbit, mouse; [Tyr⁰]-prepro ANF 104-123, human; urodilatin (CDD/ANP 95-126); ventricular natriuretic peptide (VNP), eel; and ventricular natriuretic peptide (VNP), rainbow trout.

Bag cell peptides including, but not limited to, alpha bag cell peptide; alpha-bag cell peptide 1-9; alpha-bag cell peptide 1-8; alpha-bag cell peptide 1-7; beta-bag cell factor; and gamma-bag cell factor.

Bombesin peptides including, but not limited to, alpha-s1 casein 101-123 (bovine milk); biotinyl-bombesin; bombesin 8-14; bombesin; [Leu¹³-psi (CH2NH)Leu¹⁴]-bombesin; [D-Phe⁶, Des-Met¹⁴]-bombesin 6-14 ethylamide; [DPhe¹²] bombesin; [DPhe¹², Leu¹⁴]-bombesin; [Tyr⁴]-bombesin; and [Tyr⁴, DPhe¹²]-bombesin.

Bone GLA peptides (BGP) including, but not limited to, bone GLA protein; bone GLA protein 45-49; [Glu¹⁷, Gla^{21,24}]-osteocalcin 1-49, human; myclopeptide-2 MP-2); osteocalcin 1-49 human; osteocalcin 37-49, human; and [Tyr³⁸, Phe^{42,46}] bone GLA protein 38-49, human.

Bradykinin peptides including, but not limited to, [Ala^{2,6}, des-Pro³]-bradykinin; bradykinin; bradykinin (Bowfin. Gar); bradykinin potentiating peptide; bradykinin 1-3; bradykinin 1-5; bradykinin 1-6; bradykinin 1-7; bradykinin 2-7; bradykinin 2-9; [DPhe⁷] bradykinin; [Des-Arg⁹]-bradykinin; [Des-Arg¹⁰]-Lys-bradykinin ([Des-Arg¹⁰]-kallidin); [D-N-Me-Phe⁷]-bradykinin; [Des-Arg⁹, Leu⁸]-bradykinin; Lys-bradykinin (kallidin); Lys-[Des-Arg⁹, Leu⁸]-bradykinin ([Des-Arg¹⁰, Leu⁹]-kallidin); [Lys⁰-Hyp³]-bradykinin; ovokinin; [Lys⁰, Ala³]-bradykinin; Met-Lys-bradykinin; peptide K12 bradykinin potentiating peptide; [(pCl)Phe^{5,8}]-bradykinin; T-kinin (Ile-Ser-bradykinin); [Thi^{5,8}, D-Phe⁷]-bradykinin; [Tyr⁰]-bradykinin; [Tyr⁵]-bradykinin; [Tyr⁸]-bradykinin; and kallikrein.

Brain natriuretic peptides (BNP) including, but not limited to, BNP 32, canine; BNP-like Peptide, eel; BNP-32, human; BNP-45, mouse; BNP-26, porcine; BNP-32, porcine; biotinyl-BNP-32, porcine; BNP-32, rat; biotinyl-BNP-32, rat; BNP-45 (BNP 51-95, 5K cardiac natriuretic peptide), rat; and [Tyr⁰]-BNP 1-32, human.

C-peptides including, but not limited to, C-peptide; and [Tyr⁰]-C-peptide, human.

C-type natriuretic peptides (CNP) including, but not limited to, C-type natriuretic peptide, chicken; C-type natriuretic peptide-22 (CNP-22), porcine, rat, human; C-type natriuretic peptide-53 (CNP-53), human; C-type natriuretic peptide-53 (CNP-53), porcine, rat; C-type natriuretic peptide-53 (porcine, rat) 1-29 (CNP-53 1-29); prepro-CNP 1-27, rat; prepro-CNP 30-50, porcine, rat; vasonatrin peptide (VNP); and [Tyr⁰]-C-type natriuretic peptide-22 ([Tyr⁰]-CNP-22).

Calcitonin peptides including, but not limited to, biotinyl-calcitonin, human; biotinyl-calcitonin, rat; biotinyl-calcitonin, salmon; calcitonin, chicken; calcitonin, eel; calcitonin, human; calcitonin, porcine; calcitonin, rat; calcitonin, salmon; calcitonin 1-7, human; calcitonin 8-32, salmon; katacalcin (PDN-21) (C-procalcitonin); and N-proCT (amino-terminal procalcitonin cleavage peptide), human.

Calcitonin gene related peptides (CGRP) including, but not limited to, acetyl-alpha-CGRP 19-37, human; alpha-CGRP 19-37, human; alpha-CGRP 23-37, human; biotinyl-CGRP, human; biotinyl-CGRP II, human; biotinyl-CGRP, rat; beta-CGRP, rat; biotinyl-beta-CGRP, rat; CGRP, rat; CGRP, human; calcitonin C-terminal adjacent peptide; CGRP 1-19, human; CGRP 20-37, human; CGRP 8-37, human; CGRP II, human; CGRP, rat; CGRP 8-37, rat; CGRP 29-37, rat; CGRP 30-37, rat; CGRP 31-37, rat; CGRP, 32-37, rat; CGRP 33-37, rat; CGRP 31-37, rat; ([Cys(Acm)^{2,7}]-CGRP; elcatonin; [Tyr⁰]-CGRP, human; [Tyr⁰]-CGRP II, human; [Tyr⁰]-CGRP 28-37, rat; [Tyr⁰]-CGRP, rat; and [Tyr²²]-CGRP 22-37, rat.

CART peptides including, but not limited to, CART, human; CART 55-102, human; CART, rat; and CART 55-102, rat.

Casomorphin peptides including, but not limited to, beta-casomorphin, human; beta-casomorphin 1-3; beta-casomorphin 1-3, amide; beta-casomorphin, bovine; beta-casomorphin 1-4, bovine; beta-casomorphin 1-5, bovine; beta-casomorphin 1-5, amide, bovine; beta-casomorphin 1-6, bovine; [DAla²]-beta-casomorphin 1-3, amide, bovine; [DAla², Hyp⁴, Tyr⁵]-beta-casomorphin 1-5 amide; [DAla², DPro⁴, Tyr⁵]-beta-casomorphin 1-5, amide; [DAla², Tyr⁵]-beta-casomorphin 1-5, amide, bovine; [DAla^{2,4}, Tyr⁵]-beta-casomorphin 1-5, amide, bovine; [DAla², (pCl)Phe³]-beta-casomorphin, amide, bovine; [DAla²]-beta-casomorphin 1-4, amide, bovine; [DAla²]-beta-casomorphin 1-5, bovine, [DAla²]-beta-casomorphin 1-5, amide, bovine; [DAla², Met⁵]-beta-casomorphin 1-5, bovine; [DPro²]-beta-casomorphin 1-5, amide, bovine; [DAla²]-beta-casomorphin 1-6, bovine; [DPro²]-beta-casomorphin 1-4, amide; [Des-Tyr¹]-beta-casomorphin, bovine; [DAla^{2,4}, Tyr⁵]-beta-casomorphin 1-5, amide, bovine; [DAla² (pCl)Phe³]-beta-casomorphin, amide, bovine; [DAla²]-beta-casomorphin 1-4, amide, bovine, [DAla²]-beta-casomorphin 1-5, bovine; [DAla²]-beta-casomorphin 1-5, amide, bovine; [DAla²]-casomorphin 1-5, bovine; [DPro²]-beta-casomorphin 1-5, amide, bovine; [DAla²]-beta-casomorphin 1-6, bovine; [DPro²]-beta-casomorphin 1-4, amide; [Des-Tyr¹]-beta-casomorphin, bovine; and [Val³]-beta-casomorphin 1-4, amide, bovine.

Chemotactic peptides including, but not limited to, defensin 1 (human) HNP-1 (human neutrophil peptide-1); and N-formyl-Met-Leu-Phe.

Cholecystokinin (CCK) peptides including, but not limited to, caerulein; cholecystokinin; cholecystokinin-pancreozymin; CCK-33, human; cholecystokinin octapeptide 1-4 (non-sulfated) (CCK 26-29, unsulfated); cholecystokinin octapeptide (CCK 26-33); cholecystokinin octapeptide (non-sulfated) (CCK 26-33, unsulfated); cholecystokinin heptapeptide (CCK 27-33); cholecystokinin tetrapeptide (CCK 30-33); CCK-33, porcine; CR 1 409, cholecystokinin antagonist; CCK flanking peptide (unsulfated); N-acetyl cholecystokinin, CCK 26-30, sulfated; N-acetyl cholecystokinin, CCK 26-31, sulfated; N-acetyl cholecystokinin, CCK 26-31, non-sulfated; prepro CCK fragment V-9-M; and proglumide.

Colony-stimulating factor peptides including, but not limited to, colony-stimulating factor (CSF); C-M-CSF; M-CSF; and G-CSF.

Corticortropin releasing factor (CRF) peptides including, but not limited to, astressin; alpha-helical CRF 12-41; biotinyl-CRF, ovine; biotinyl-CRF, human, rat; CRF, bovine; CRF, human, rat; CRF, ovine; CRF, porcine; [Cys²¹]-CRF, human, rat; CRF antagonist (alpha-helical CRF 941); CRF-6-33, human, rat; [DPro⁵]-CRF, human, rat; [D-Phe¹², Nle^{21,38}]-CRF 12-41, human, rat; eosinophilotactic peptide; [Met(0)²¹]-CRF, ovine; [Nle²¹, Tyr³²]-CRF, ovine; prepro CRF 125-151, human; sauvagine, frog; [Tyr⁰]-CRF, human, rat; [Tyr⁰]-CRF, ovine; [Tyr⁰]-CRF 34-41, ovine; [Tyr⁰]-urocortin; urocortin amide, human; urocortin, rat; urotensin I (Catostomus commersoni); urotensin II; and urotensin II (Rana ridibunda).

Cortistatin peptides including, but not limited to, cortistatin 29; cortistatin 29 (1-13); [Tyr⁰]-cortistatin 29; pro-cortistatin 28-47; and pro-cortistatin 51-81.

Cytokine peptides including, but not limited to, tumor necrosis factor alpha (TNF-α); and tumor necrosis factor-β (TNF-β). Interleukins, including but not limited to IL-1α, IL-1β, IL-2, IL-3, IL-4, IL-5, IL-6, IL-7, IL-8, IL-10, IL-12, and IL-13. Interleukin peptides including, but not limited to, interleukin-1 beta 165-181, rat; and interleukin-8 (IL-8, CINC/gro), rat. Chemokines including but not limited to RANTES, MCP-1, MIP-1α, MIP-1β.

Dermorphin peptides including, but not limited to, dermorphin and dermorphin analog 1-4.

Dynorphin peptides including, but not limited to, big dynorphin (prodynorphin 209-240), porcine; biotinyl-dynorphin A (biotinyl-prodynorphin 209-225); [DAla², DArg⁶]-dynorphin A 1-13, porcine; [D-Ala²]-dynorphin A, porcine; [D-Ala²]-dynorphin A amide, porcine; [D-Ala²]-dynorphin A 1-13, amide, porcine; [D-Ala²]-dynorphin A 1-9, porcine; [DArg⁶]-dynorphin A 1-13, porcine; [DArg⁸]-dynorphin A 1-13, porcine; [Des-Tyr¹]-dynorphin A 1-8; [D-Pro¹⁰]-dynorphin A 1-11, porcine; dynorphin A amide, porcine; dynorphin A 1-6, porcine; dynorphin A 1-7, porcine; dynorphin A 1-8, porcine; dynorphin A 1-9, porcine; dynorphin A 1-10, porcine; dynorphin A 1-10 amide, porcine; dynorphin A 1-11, porcine; dynorphin A 1-12, porcine; dynorphin A 1-13, porcine; dynorphin A 1-13 amide, porcine; DAKLI (dynorphin A-analogue kappa ligand); DAKLI-biotin ([Arg^{11,13}]-dynorphin A (1-13)-Gly-NH(CH2)5NH-biotin); dynorphin A 2-17, porcine; dynorphin 2-17, amide, porcine; dynorphin A 2-12, porcine; dynorphin A 3-17, amide, porcine; dynorphin A 3-8, porcine; dynorphin A 3-13, porcine; dynorphin A 3-17, porcine; dynorphin A 7-17, porcine; dynorphin A 8-17, porcine; dynorphin A 6-17, porcine; dynorphin A 13-17, porcine; dynorphin A (prodynorphin 209-225), porcine; dynorphin B 1-9; [MeTyr¹, MeArg⁷, D-Leu⁸]-dynorphin 1-8 ethyl amide; [(nMe)Tyr¹] dynorphin A 1-13, amide, porcine; [Phe⁷]-dynorphin A 1-7, porcine; [Phe⁷]-dynorphin A 1-7, amide, porcine; and prodynorphin 228-256 (dynorphin B 29) (leumorphin), porcine.

Endorphin peptides including, but not limited to, alpha-neo-endorphin, porcine; beta-neo-endorphin; Ac-beta-endorphin, camel, bovine, ovine; Ac-beta-endorphin 1-27, camel, bovine, ovine; Ac-beta-endorphin, human; Ac-beta-endorphin 1-26, human; Ac-beta-endorphin 1-27, human; Ac-gamma-endorphin (Ac-beta-lipotropin 61-77); acetyl-alpha-endorphin; alpha-endorphin (beta-lipotropin 61-76); alpha-neo-endorphin analog; alpha-neo-endorphin 1-7; [Arg⁸]-alpha-neo-endorphin 1-8; beta-endorphin (beta-lipotropin 61-91), camel, bovine, ovine; beta-endorphin 1-27, camel, bovine, ovine; beta-endorphin, equine, beta-endorphin (beta-lipotropin 61-91), human; beta-endorphin (1-5)+(16-31), human; beta-endorphin 1-26, human; beta-endorphin 1-27, human; beta-endorphin 6-31, human; beta-endorphin 18-31, human; beta-endorphin, porcine; beta-endorphin, rat; beta-lipotropin 1-10, porcine; beta-lipotropin 60-65; beta-lipotropin 61-64; beta-lipotropin 61-69; beta-lipotropin 88-91; biotinyl-beta-endorphin (biotinyl-beta-lipotropin 61-91); biocytin-beta-endorphin, human; gamma-endorphin (beta-lipotropin 61-77); [DAla²]-alpha-neo-endorphin 1-2, amide; [DAla²]-beta-lipotropin 61-69; [DAla²]-gamma-endorphin; [Des-Tyr¹]-beta-endorphin, human; [Des-Tyr¹]-gamma-endorphin (beta-lipotropin 62-77); [Leu⁵]-beta-endorphin, camel, bovine, ovine; [Met⁵, Lys⁶]-alpha-neo-endorphin 1-6; [Met⁵, Lys^{6,7}]-alpha-neo-endorphin 1-7; and [Met⁵, Lys⁶, Arg⁷]-alpha-neo-endorphin 1-7.

Endothelin peptides including, but not limited to, endothelin-1 (ET-1); endothelin-1[Biotin-Lys⁹]; endothelin-1 (1-15), human; endothelin-1 (1-15), amide, human; Ac-endothelin-1 (16-21), human; Ac-[DTrp¹⁶]-endothelin-1 (16-21), human; [Ala^{3,11}]-endothelin-1; [Dpr1, Asp¹⁵]-endothelin-1; [Ala²]-endothelin-3, human; [Ala¹⁸]-endothelin-1, human; [Asn¹⁸]-endothelin-1, human; [Res-701-1]-endothelin B receptor antagonist; Suc-[Glu⁹, Ala^{11,15}]-endothelin-1 (8-21), IRL-1620; endothelin-C-terminal hexapeptide: [D-Val²²]-big endothelin-1 (16-38), human; endothelin-2 (ET-2), human, canine; endothelin-3 (ET-3), human, rat, porcine, rabbit; biotinyl-endothelin-3 (biotinyl-ET-3); prepro-endothelin-1 (94-109), porcine; BQ-518; BQ-610; BQ-788; endothelium-dependent relaxation antagonist; FR139317; IRL-1038; JKC-30 1; JKC-302; PD-145065; PD 142893; sarafotoxin S6a (atractaspis engaddensis); sarafotoxin S6b (atractaspis engaddensis); sarafotoxin S6c (atractaspis engaddensis); [Lys⁴]-sarafotoxin S6c; sarafotoxin S6d; big endothelin-1, human; biotinyl-big endothelin-1, human; big endothelin-1 (1-39), porcine; big endothelin-3 (22-41), amide, human; big endothelin-1 (22-39), rat; big endothelin-1 (1-39), bovine; big endothelin-1 (22-39), bovine; big endothelin-1 (19-38), human; big endothelin-1 (22-38), human; big endothelin-2, human; big endothelin-2 (22-37), human; big endothelin-3, human; big endothelin-1, porcine; big endothelin-1 (22-39) (prepro-endothelin-1 (74-91)); big endothelin-1, rat; big endothelin-2 (1-38), human; big endothelin-2 (22-38), human; big endothelin-3, rat; biotinyl-big endothelin-1, human; and [Tyr¹²³]-prepro-endothelin (110-130), amide, human.

ETa receptor antagonist peptides including, but not limited to, [BQ-123]; [BE18257B]; [BE-18257A]/[W-7338A]; [BQ-485]; FR139317; PD-151242; and TTA-386.

ETb receptor antagonist peptides including, but not limited to, [BQ-3020]; [RES-701-3]; and [IRL-1720].

Enkephalin peptides including, but not limited to, adrenorphin, free acid; amidorphin (proenkephalin A (104-129)-NH2), bovine; BAM-12P (bovine adrenal medulla dodecapeptide); BAM-22P (bovine adrenal medulla docosapeptide); benzoyl-Phe-Ala-Arg; enkephalin; [D-Ala², D-Leu⁵]-enkephalin; [D-Ala², D-Met⁵]-enkephalin; [DAla²]-Leu-enkephalin, amide; [DAla², Leu⁵, Arg⁶]-enkephalin; [Des-Tyr¹, DPen^{2,5}]-enkephalin; [(Des-Tyr¹, DPen², Pen⁵]-enkephalin; [Des-Tyr¹]-Leu-enkephalin; [D-Pen^{2,5}]-enkephalin; [DPen², Pen⁵]-enkephalin; enkephalinase substrate; [D-Pen², pCl-Phe⁴, D-Pen⁵]-enkephalin; Leu-enkephalin; Leu-enkephalin, amide; biotinyl-Leu-enkephalin; [D-Ala²]-Leu-enkephalin; [D-Ser²]-Leu-enkephalin-Thr (delta-receptor peptide) (DSLET); [D-Thr²]-Leu-enkephalin-Thr (DTLET); [Lys⁶]-Leu-enkephalin, [Met⁵, Arg⁶]-enkephalin; [Met⁵, Arg⁶]-enkephalin-Arg; [Met⁵, Arg⁶, Phe⁷]-enkephalin, amide; Met-enkephalin; biotinyl-Met-enkephalin; [D-Ala²]-Met-enkephalin; [D-Ala²]-Met-enkephalin, amide; Met-enkephalin-Arg-Phe; Met-enkephalin, amide; [Ala²]-Met-enkephalin, amide; [DMet², Pro⁵]-enkephalin, amide; [DTrp²]-Met-enkephalin, amide, metorphinamide (adrenorphin); peptide B, bovine; 3200-Dalton adrenal peptide E, bovine; peptide F, bovine; preproenkephalin B 186-204, human; spinorphin, bovine; and thiorphan (D, L, 3-mercapto-2-benzylpropanoyl-glycine).

Ephrin B, its Analogues and Antagonists.

Fibronectin peptides including, but not limited to platelet factor-4 (58-70), human; echistatin (Echis carinatus); E, P, L selectin conserved region; fibronectin analog; fibronectin-binding protein; fibrinopeptide A, human; [Tyr⁰]-fibrinopeptide A, human; fibrinopeptide B, human; [Glu¹]-fibrinopeptide B, human; [Tyr¹⁵]-fibrinopeptide B, human; fibrinogen beta-chain fragment of 24-42; fibrinogen binding inhibitor peptide; fibronectin related peptide (collagen binding fragment); fibrinolysis inhibiting factor, FN-C/H-1 (fibronectin heparin-binding fragment); FN-C/H-V (fibronectin heparin-binding fragment); heparin-binding peptide; laminin penta peptide, amide; Leu-Asp-Val-NH2 (LDV-NH2), human, bovine, rat, chicken; necrofibrin, human; necrofibrin, rat; and platelet membrane glycoprotein IIB peptide 296-306.

Galanin peptides including, but not limited to, galanin, human; galanin 1-19, human; preprogalanin 1-30, human; preprogalanin 65-88, human, preprogalanin 89-123, human; galanin, porcine; galanin 1-16, porcine, rat; galanin, rat; biotinyl-galanin, rat; preprogalanin 28-67, rat; galanin 1-13-bradykinin 2-9, amide; M40, galanin 1-13-Pro-Pro-(Ala-Leu) 2-Ala-amide; C7, galanin 1-13-spanitide-amide; GMAP 1-41, amide; GMAP 16-41, amide; GMAP 25-41, amide; galantide; and entero-kassinin.

Gastrin peptides including, but not limited to, gastrin, chicken; gastric inhibitory peptide (GIP), human; gastrin 1, human; biotinyl-gastrin 1, human; big gastrin-1, human; gastrin releasing peptide, human; gastrin releasing peptide 1-16, human; gastric inhibitory polypeptide (GIP), porcine; gastrin releasing peptide, porcine; biotinyl-gastrin releasing peptide, porcine; gastrin releasing peptide 14-27, porcine, human; little gastrin, rat; pentagastrin; gastric inhibitory peptide 1-30, porcine; gastric inhibitory peptide 1-30, amide, porcine; [Tyr⁰]-gastric inhibitory peptide 23-42, human; and gastric inhibitory peptide, rat.

Glucagon peptides including, but not limited to, [Des-His¹, Glu⁹]-glucagon, extendin-4, glucagon, human; biotinyl-glucagon, human; glucagon 19-29, human; glucagon 22-29, human; Des-His¹-[Glu⁹]-glucagon, amide; glucagon-like peptide 1, amide (preproglucagon 72-107, amide); glucagon-like peptide I (preproglucagon 72-108), human; glucagon-like peptide 1 (7-36) (preproglucagon 78-107, amide); glucagon-like peptide II, rat; biotinyl-glucagon-like peptide-1 (7-36) (biotinyl-preproglucagon 78-107, amide); glucagon-like peptide 2 (preproglucagon 126-159), human; oxyntomodulin/glucagon 37; and valosin (peptide VQY), porcine.

Gn-RH associated peptides (GAP) including, but not limited to, Gn-RH associated peptide 25-53, human; Gn-RH associated peptide 1-24, human; Gn-RH associated peptide 1-13, human; Gn-RH associated peptide 1-13, rat; gonadotropin releasing peptide, follicular, human; [Tyr⁰]-GAP ([Tyr⁰]-Gn-RH Precursor Peptide 14-69), human; and proopiomelanocortin (POMC) precursor 27-52, porcine.

Growth factor peptides including, but not limited to, cell growth factors; epidermal growth factors; tumor growth factor; TGF-alpha, human; TGF-alpha, from other mammalian species TGF-beta; alpha-TGF 34-43, human EGF (epidermal growth factor); acidic fibroblast growth factor; basic fibroblast growth factor; basic fibroblast growth factor 13-18; basic fibroblast growth factor 120-125; brain derived acidic fibroblast growth factor 1-11; brain derived basic fibroblast growth factor 1-24; brain derived acidic fibroblast growth factor 102-111; [Cys(Acm^{20,31})]-epidermal growth factor 20-31; epidermal growth factor receptor peptide 985-996; insulin-like growth factor (IGF)-I, chicken; IGF-I, rat; IGF-I, human; Des (1-3) IGF-I, human; R3 IGF-I, human; R3 IGF-I, human; long R3 IGF-I, human; adjuvant peptide analog; anorexigenic peptide; Des (1-6) IGF-II, human; R6 IGF-II, human; IGF-I analogue; IGF I (24-41); IGF I (57-70); IGF I (30-41); IGF II; IGF II (33-40); [Tyr⁰]-IGF II (33-40); liver cell growth factor; midkine; midkine 60-121, human; N-acetyl, alpha-TGF 34-43, methyl ester, rat; nerve growth factor (NGF), mouse; platelet-derived growth factor; platelet-derived growth factor antagonist; ligands for the receptors of the Erb-B family.

Growth hormone peptides including, but not limited to, growth hormone (hGH), human; growth hormone 1-43, human; growth hormone 6-13, human; growth hormone releasing factor, human; growth hormone releasing factor, bovine; growth hormone releasing factor, porcine; growth hormone releasing factor 1-29, amide, rat; growth hormone pro-releasing factor, human; biotinyl-growth hormone releasing factor, human; growth hormone releasing factor 1-29, amide, human; [D-Ala²]-growth hormone releasing factor 1-29, amide, human; [N-Ac-Tyr¹, D-Arg²]-GRF 1-29, amide; [His¹, Nle²⁷]-growth hormone releasing factor 1-32, amide; growth hormone releasing factor 1-37, human; growth hormone releasing factor 1-40, human; growth hormone releasing factor 1-40, amide, human; growth hormone releasing factor 30-44, amide, human; growth hormone releasing factor, mouse; growth hormone releasing factor, ovine; growth hormone releasing factor, rat; biotinyl-growth hormone releasing factor, rat; GHRP-6 ([His¹, Lys⁶]-GHRP); hexarelin (growth hormone releasing hexapeptide); and [D-Lys³]-GHRP-6.

GTP-binding proteins and fragment peptides thereof including, but not limited to, [Arg⁸]-GTP-binding protein fragment, Gs alpha; GTP-binding protein fragments, of the G beta family; GTP-binding protein fragments, of the Ggamma family; GTP-binding protein fragment, Galpha; GTP-binding protein fragments, Go alpha a and b; GTP-binding protein fragment, Gs alpha; and GTP-binding protein fragments, G alpha i1, G alpha i2, G alpha i3; GTP-binding protein fragment, Golf alpha; GTP-binding protein fragment, Gz alpha; GTP-binding protein fragment, Gq alpha.

Guanylin peptides including, but not limited to, guanylin, human; guanylin, rat; and uroguanylin.

Inhibin peptides including, but not limited to, inhibin, bovine; inhibin, alpha-subunit 1-32, human; [Tyr⁰]-inhibin, alpha-subunit 1-32, human; seminal plasma inhibin-like peptide, human; [Tyr⁰]-seminal plasma inhibin-like peptide, human; inhibin, alpha-subunit 1-32, porcine; and [Tyr⁰]-inhibin, alpha-subunit 1-32, porcine.

Interferon peptides including, but not limited to, alpha interferon species (e.g., alpha1, alpha2, alpha2a, alpha2b, alpha2c, alpha2d, alpha3, alpha4, alpha4a, alpha4b, alpha5, alpha6, alpha74, alpha76, alphaA, alphaB, alphaC, alphaC1, alphaD, alphaE, alphaF, alphaG, alphaG, alphaH, alphaI, alphaJ1, alphaJ2, alphaK, alphaL); interferon beta species (e.g., beta1a); interferon gamma species (e.g., gamma1a, gamma1b); interferon epsilon; interferon tau; interferon omega or any analogues of interferon omega. Various analogs of gamma interferon are described in Pechenov et al. “Methods for preparation of recombinant cytokine proteins V. mutant analogues of human interferon-gamma with higher stability and activity” Protein Expr. Purif. 24:173-180 (2002), which is incorporated herein by reference in its entirety for teachings directed to preparation and testing of interferon analogues.

Insulin peptides including, but not limited to, insulin, human; insulin, porcine; IGF-I, human; insulin-like growth factor II (69-84); pro-insulin-like growth factor II (68-102), human; pro-insulin-like growth factor II (105-128), human; [Asp^{B28}]-insulin, human; [Lys^{B28}]-insulin, human; [Leu^{B28}]-insulin, human; [Val^{B28}]-insulin, human; [Ala^{B28}]-insulin, human; [Asp^{B28}, Pro^{B29}]-insulin, human; [Lys^{B28}, Pro^{B29}]-insulin, human; [Leu^{B28}, Pro^{B29}]-insulin, human; [Val^{B28}, Pro^{B28}]-insulin, human; [Ala^{B28}, Pro^{B28}]-insulin, human; [Gly^{A21}]-insulin, human; [Gly^{A21} Gln^{B3}]-insulin, human; [Ala^{A21}]-insulin, human; [Ala^{A}21 Gln^{B3}]-insulin, human; [Gln^{B3}]-insulin, human; [Gln^{B30}]-insulin, human; [Gly^{A21} Glu^{B30}]-insulin, human; [Gly^{A21} Gln^{B3} Glu^{B30}]-insulin, human; [Gln^{B3} Glu^{B30}]-insulin, human; B22-B30 insulin, human; B23-B30 insulin, human; B25-B30 insulin, human; B26-B30 insulin, human; B27-B30 insulin, human; B29-B30 insulin, human; the A chain of human insulin, and the B chain of human insulin.

Laminin peptides including, but not limited to, laminin; alpha1 (I)-CB3 435-438, rat; and laminin binding inhibitor.

Leptin peptides including, but not limited to, leptin 93-105, human; leptin 22-56, rat; Tyr-leptin 26-39, human; and leptin 116-130, amide, mouse.

Leucokinin peptides including, but not limited to, leucomyosuppressin (LMS); leucopyrokinin (LPK); leucokinin I; leucokinin II; leucokinin III; leucokinin IV; leucokinin VI; leucokinin VII; and leucokinin VIII.

Luteinizing hormone-releasing hormone peptides including, but not limited to, antide; Gn-RH II, chicken; luteinizing hormone-releasing hormone (LH-RH) (GnRH); biotinyl-LH-RH; cetrorelix (D-20761); [D-Ala⁶]-LH-RH; [Gln⁸]-LH-RH (Chicken LH-RH); [DLeu⁶, Val⁷] LH-RH 1-9, ethyl amide; [D-Lys⁶]-LH-RH; [D-Phe², Pro³, D-Phe⁶]-LH-RH; [DPhe², DAla⁶] LH-RH; [Des-Gly¹⁰]-LH-RH, ethyl amide; [D-Ala⁶, Des-Gly¹⁰]-LH-RH, ethyl amide; [DTrp⁶]-LH-RH, ethyl amide; [D-Trp⁶, Des-Gly¹⁰]-LH-RH, ethyl amide (Deslorelin); [DSer(But)⁶, Des-Gly¹⁰]-LH-RH, ethyl amide; ethyl amide; leuprolide; LH-RH 4-10; LH-RH 7-10; LH-RH, free acid; LH-RH, lamprey; LH-RH, salmon; [Lys⁸]-LH-RH; [Trp⁷, Leu⁸] LH-RH, free acid; and [(t-Bu)DSer⁶, (Aza)Gly¹⁰]-LH-RH.

Mastoparan peptides including, but not limited to, mastoparan; mas7; mas8; mas17; and mastoparan X.

Mast cell degranulating peptides including, but not limited to, mast cell degranulating peptide HR-1; and mast cell degranulating peptide HR-2.

Melanocyte stimulating hormone (MSH) peptides including, but not limited to, [Ac-Cys⁴, DPhe⁷, Cys¹⁰] alpha-MSH 4-13, amide; alpha-melanocyte stimulating hormone; alpha-MSH, free acid; beta-MSH, porcine; biotinyl-alpha-melanocyte stimulating hormone; biotinyl-[Nle⁴, D-Phe⁷] alpha-melanocyte stimulating hormone; [Des-Acetyl]-alpha-MSH; [DPhe⁷]-alpha-MSH, amide; gamma-1-MSH, amide; [Lys⁰]-gamma-1-MSH, amide; MSH release inhibiting factor, amide; [Nle⁴]-alpha-MSH, amide; [Nle⁴, D-Phe⁷]-alpha-MSH; N-Acetyl, [Nle⁴, DPhe⁷] alpha-MSH 4-10, amide; beta-MSH, human; and gamma-MSH.

Morphiceptin peptides including, but not limited to, morphiceptin (beta-casomorphin 1-4 amide); [D-Pro⁴]-morphiceptin; and [N-MePhe³, D-Pro⁴]-morphiceptin.

Motilin peptides including, but not limited to, motilin, canine; motilin, porcine; biotinyl-motilin, porcine; and [Leu¹³]-motilin, porcine.

Neuro-peptides including, but not limited to, Ac-Asp-Glu; achatina cardioexcitatory peptide-1 (ACEP-1) (Achatina fulica); adipokinetic hormone (AKH) (Locust); adipokinetic hormone (Heliothis zea and Manduca sexta); alytesin; Tabanus atratus adipokinctic hormone (Taa-AKH); adipokinetic hormone II (Locusta migratoria); adipokinetic hormone II (Schistocera gregaria); adipokinetic hormone III (AKH-3); adipokinetic hormone G (AKH-G) (Gryllus bimaculatus); allatotropin (AT) (Manduca sexta); allatotropin 6-13 (Manduca sexta); APGW amide (Lymnaea stagnalis); buccalin; cerebellin; [Des-Ser¹]-cerebellin; corazonin (American Cockroach Periplaneta americana); crustacean cardioactive peptide (CCAP); crustacean erythiophiore; DF2 (Procambarus clarkii); diazepam-binding inhibitor fragment, human; diazepam binding inhibitor fragment (ODN); eledoisin related peptide; FMRF amide (molluscan cardioexcitatory neuro-peptide); Gly-Pro-Glu (GPE), human; granuliberin R; head activator neuropeptide; [His⁷]-corazonin; stick insect hypertrehalosacmic factor II; Tabanus atratos hypotrehalosemic hormone (Taa-HoTH); isoguvacine hydrochloride; bicuculline methiodide; piperidine-4-sulphonic acid; joining peptide of proopiomelanocortin (POMC), bovine; joining peptide, rat; KSAYMRF amide (P. redivivus); kassinin; kinetensin; levitide; litorin; LUQ 81-91 (Aplysia californica); LUQ 83-91 (Aplysia californica); myoactive peptide I (Periplanetin CC-1) (Neuro-hormone D); myoactive peptide II (Peripianetin CC-2); myomodulin; neuron specific peptide; neuron specific enolase 404-443, rat; neuropeptide FF; neuropeptide K, porcine; NEI (prepro-MCH 131-143) neuropeptide, rat; NGE (prepro-MCH 110-128) neuropeptide, rat; NFI (Procambarus clarkii); PBAN-1 (Bombyx mori); Hez-PBAN (Heliothis zea); SCPB (cardioactive peptide from aplysia); secretoneurin, rat; uperolein; urechistachykinin I; urechistachykinin II; xenopsin-related peptide I; xenopsin-related peptide II; pedal peptide (Pep), aplysia; peptide FI, lobster; phyllomedusin; polistes nmastoparan; proctolin; ranatensin; Ro I (Lubber Grasshopper, Romalea microptera); Ro II (Lubber Grasshopper, Romalea microptera); SALMF amide 1 (S1); SALMF amide 2 (S2); and SCPA.

Neuropeptide Y (NPY) peptides including, but not limited to, [Leu³¹, Pro³⁴]-neuropeptide Y, human; neuropeptide F (Moniezia expansa); B1BP3226 NPY antagonist; Bis (31/31′) {[Cys³¹, Trp³²; Nva³⁴] NPY 31-36}; neuropeptide Y, human, rat; neuropeptide Y 1-24 amide, human; biotinyl-neuropeptide Y; [D-Tyr^{27,36}, D-Thr³²]-NPY 27-36; Des 10-17 (cyclo 7-21) [Cys^{7,21}, Pro³⁴]-NPY; C2-NPY; [Leu³¹, Pro³⁴] neuropeptide Y, human; neuropeptide Y, free acid, human; neuropeptide Y, free acid, porcine; prepro NPY 68-97, human; N-acetyl-[Leu²⁸, Leu³¹] NPY 24-36; neuropeptide Y, porcine; [D-Trp³²]-neuropeptide Y, porcine; [D-Trp³²] NPY 1-36, human; [Leu¹⁷, DTrp³²] neuropeptide Y, human; [Leu³¹, Pro³⁴]-NPY, porcine; NPY 2-36, porcine; NPY 3-36, human; NPY 3-36, porcine; NPY 13-36, human; NPY 13-36, porcine; NPY 16-36 porcine; NPY 18-36, porcine; NPY 20-36; NPY 22-36; NPY 26-36; [Pro³⁴]-NPY 1-36, human; [Pro³⁴]-neuropeptide Y, porcine; PYX-1; PYX-2; T4-[NPY(33-36)]4; and Tyr(OMe)²¹]-neuropeptide Y, human.

Neurotropic factor peptides including, but not limited to, glial derived neurotropic factor (GDNF); brain derived neurotropic factor (BDNF); and ciliary neurotropic factor (CNTF).

Ligands of the Notch receptor including, but not limited to the Delta-like-1, Delta-like-2, Delta-like-3, Delta-like-4, Jagged-1 and Jagged-2 proteins, and fragments thereof.

Orexin peptides including, but not limited to, orexin A; orexin B, human; orexin B, rat, mouse.

Opioid peptides including, but not limited to, alpha-casein fragment 90-95; BAM-18P; casomokinin L; casoxin D; crystalline; DALDA; dermenkephalin (deltorphin) (Phylomedusa sauvagei); [D-Ala²]-deltorphin I; [D-Ala²]-deltorphin II; endomorphin-1; endomorphin-2; kyotorphin; [DArg²]-kyotorphin; morphin tolerance peptide; morphine modulating peptide, C-terminal fragment; morphine modulating neuropeptide (A-18-F-NH2); nociceptin [orphanin FQ] (ORL1 agonist); TIPP; Tyr-MIF-1; Tyr-W-MIF-1; valorphin; LW-hemorphin-6, human; Leu-valorphin-Arg; and Z-Pro-D-Leu.

Oxytocin peptides including, but not limited to, [Asu⁶]-oxytocin; oxytocin; biotinyl-oxytocin; [Thr⁴, Gly⁷]-oxytocin; and tocinoic acid ([Ile³]-pressinoic acid).

PACAP (pituitary adenylating cyclase activating peptide) peptides including, but not limited to, PACAP 1-27, human, ovine, rat; PACAP (1-27)-Gly-Lys-Arg-NH2, human; [Des-Gln¹⁶]-PACAP 6-27, human, ovine, rat; PACAP38, frog; PACAP27-NH2, human, ovine, rat, biotinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP27-NH2, human; ovine, rat; biotinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat: PACAP38 16-38, human, ovine, rat; PACAP38 31-38, human, ovine, rat; PACAP38 31-38, human, ovine, rat; PACAP-related peptide (PRP), human; and PACAP-related peptide (PRP), rat.

Pancreastatin peptides including, but not limited to, chromostatin, bovine; pancreastatin (hPST-52) (chromogranin A 250-301, amide); pancreastatin 24-52 (hPST-29), human; chromogranin A 286-301, amide, human; pancreastatin, porcine; biotinyl-pancreastatin, porcine; [Nle⁸]-pancreastatin, porcine; [Tyr⁰, Nle⁸]-pancreastatin, porcine; [Tyr⁰]-pancreastatin, porcine; parastatin 1-19 (chromogranin A 347-365), porcine; pancreastatin (chromogranin A 264-314-amide, rat; biotinyl-pancreastatin (biotinyl-chromogranin A 264-314-amide; [Tyr⁰]-pancreastatin, rat; pancreastatin 26-51, rat; and pancreastatin 33-49, porcine.

Pancreatic polypeptides including, but not limited to, pancreatic polypeptide, avian; pancreatic polypeptide, human; C-fragment pancreatic polypeptide acid, human; C-fragment pancreatic polypeptide amide, human; pancreatic polypeptide (Rana temporaria); pancreatic polypeptide, rat; and pancreatic polypeptide, salmon.

Parathyroid hormone peptides including, but not limited to, [Asp⁷⁶]-parathyroid hormone 39-84, human; [Asp⁷⁶]-parathyroid hormone 53-84, human; [Asn⁷⁶]-parathyroid hormone 1-84, hormone; [Asn⁷⁶]-parathyroid hormone 64-84, human; [Asn⁸, Leu¹⁸]-parathyroid hormone 1-34, human; [Cys^{5,28}]-parathyroid hormone 1-34, human; hypercalcemia malignancy factor 1-40; [Leu¹⁸]-parathyroid hormone 1-34, human; [Lys(biotinyl)¹³, Nle^{8,18}, Tyr³⁴]-parathyroid hormone 1-34 amide; [Nle^{8,18}, Tyr³⁴]-parathyroid hormone 1-34 amide, [Nle^{8,18}, Tyr³⁴]-parathyroid hormone 3-34 amide, bovine; [Nle^{8,18}, Tyr³⁴]-parathyroid hormone 1-34, human; [Nle^{8,18}, Tyr³⁴]-parathyroid hormone 1-34 amide, human; [Nle^{8,18}, Tyr³⁴]-parathyroid hormone 3-34 amide, human; [Nle^{8,18}, Tyr³⁴]-parathyroid hormone 7-34 amide, bovine; [Nle^{8,21}, Tyr³⁴]-parathyroid hormone 1-34 amide, rat; parathyroid hormone 44-68, human; parathyroid hormone 1-34, bovine; parathyroid hormone 3-34, bovine parathyroid hormone 1-31 amide, human; parathyroid hormone 1-34, human; parathyroid hormone 13-34, human; parathyroid hormone 1-34, rat; parathyroid hormone 1-38, human; parathyroid hormone 1-44, human; parathyroid hormone 28-48, human; parathyroid hormone 39-68, human; parathyroid hormone 39-84, human, parathyroid hormone 53-84, human; parathyroid hormone 69-84, human; parathyroid hormone 70-84, human; [Pro³⁴]-peptide YY (PYY), human; [Tyr⁰]-hypercalcemia malignancy factor 1-40; [Tyr⁰]-parathyroid hormone 1-44, human; [Tyr⁰]-parathyroid hormone 1-34, human; [Tyr⁰]-parathyroid hormone 1-34, human; [Tyr²⁷]-parathyroid hormone 27-48, human; [Tyr³⁴]-parathyroid hormone 7-34 amide, bovine; [Tyr⁴³]-parathyroid hormone 43-68, human; [Tyr⁵², Asn⁷⁶]-parathyroid hormone 52-84, human; and [Tyr⁶³]-parathyroid hormone 63-84, human.

Parathyroid hormone (PTH)-related peptides including, but not limited to, PTHrP ([Tyr³⁶]-PTHrP 1-36 amide), chicken; hHCF-(1-34)-NH2 (humoral hypercalcemic factor), human; PTH-related protein 1-34, human; biotinyl-PTH-related protein 1-34, human; [Tyr⁰]-PTH-related protein 1-34, human; [Tyr³⁴]-PTH-related protein 1-34 amide, human; PTH-related protein 1-37, human; PTH-related protein 7-34 amide, 2746 human; PTH-related protein 38-64 amide, human; PTH-related protein 67-86 amide, human; PTH-related protein 107-111, human, rat, mouse; PTH-related protein 107-111 free acid; PTH-related protein 107-138, human; and PTH-related protein 109-111, human.

Peptide T peptides including, but not limited to, peptide T; [D-Ala¹]-peptide T; and [D-Ala¹]-peptide T amide.

Prolactin-releasing peptides including, but not limited to, prolactin-releasing peptide 31, human; prolactin-releasing peptide 20, human; prolactin-releasing peptide 31, rat; prolactin-releasing peptide 20, rat; prolactin-releasing peptide 31, bovine; and prolactin-releasing peptide 20, bovine.

Peptide YY (PYY) peptides including, but not limited to, PYY, human; PYY 3-36, human; biotinyl-PYY, human; PYY, porcine, rat; and [Leu³¹, Pro³⁴]-PYY, human.

Renin substrate peptides including, but not limited to, acetyl, angiotensinogen 1-14, human; angiotensinogen 1-14, porcine; renin substrate tetradecapeptide, rat; [Cys⁸]-renin substrate tetradecapeptide, rat; [Leu⁸]-renin substrate tetradecapeptide, rat; and [Val⁸]-renin substrate tetradecapeptide, rat.

Secretin peptides including, but not limited to, secretin, canine; secretin, chicken; secretin, human; biotinyl-secretin, human; secretin, porcine; and secretin, rat.

Somalostatin (GIF) peptides including, but not limited to, BIM-23027; biotinyl-somatostatin; biotinylated cortistatin 17, human; cortistatin 14, rat; cortistatin 17, human; [Tyr⁰]-cortistatin 17, human; cortistatin 29, rat; [D-Trp⁸]-somatostatin; [DTrp⁸, DCys¹⁴]-somatostatin; [DTrp⁸, Tyr¹¹]-somatostatin; [D-Trp¹¹]-somatostatin; NTB (Naltriben); [Nle⁸]-somatostatin 1-28; octreotide (SMS 201-995); prosomatostatin 1-32, porcine: [Tyr⁰]-somatostatin; [Tyr¹]-somatostatin; [Tyr¹]-somatostatin 28 (1-14); [Tyr¹¹]-somatostatin; [Tyr⁰, D-Trp⁸]-somatostatin; somatostatin; somatostatin antagonist; somatostatin-25; somatostatin-28; somatostatin 28 (1-12); biotinyl-somatostatin-28; [Tyr⁰]-somatostatin-28: [Leu⁸, D-Trp²², Tyr²⁵]-somatostatin-28; biotinyl-[Leu⁸, D-Trp²², Tyr²⁵]-somatostatin-28; somatostatin-28 (1-14); and somatostatin analog, RC-160.

Substance P peptides including, but not limited to, G protein antagonist-2; Ac-[Arg⁶, Sar⁹, Met(02)¹¹]-substance P 6-11; [Arg³]-substance P; Ac-Trp-3,5-bis(trifluoromethyl) benzyl ester, Ac-[Arg⁶, Sar⁹, Met(O2)¹¹]-substance P 6-11; [D-Ala⁴]-substance P 4-11; [Tyr⁶, D-Phe⁷, D-His⁹]-substance P 6-11 (sendide); biotinyl-substance P; biotinyl-NTE[Arg³]-substance P; [Tyr⁸]-substance P; [Sar⁹, Met(O2)¹¹]-substance P; [D-Pro², D-Trp^{7,9}]-substance P; [D-Pro⁴, 0-Trp^{7,9}]-substance P 4-11; substance P 4-11; [DTrp^{2,7,9}]-substance P; [(Dehydro)Pro^{2,4}, Pro⁹]-substance P; [Dehydro-Pro⁴]-substance P 4-11; [Glp⁵, (Me)Phe⁸, Sar⁹]-substance P 5-11; [Glp⁵, Sar⁹]-substance P 5-11; [Glp⁵]-substance P 5-11; hepta-substance P (substance P 5-11); hexa-substance P (substance P 6-11); [MePhe⁸, Sar⁹]-substance P; [Nle¹¹]-substance P; Octa-substance P (substance P 4-11); [pGlu¹]-hexa-substance P ([pGlu⁶]-substance P 6-11); [pGlu⁶, D-Pro⁹]-substance P 6-11; [(pNO2)Phe⁷Nle¹¹]-substance P; penta-substance P (substance P 7-11); [Pro⁹]-substance P; GR73632, substance P 7-11; [Sar⁴]-substance P 4-11; [Sar⁹]-substance P; septide ([pGlu⁶, Pro⁴]-substance P 6-11); spantide I; spantide II; substance P; substance P, cod; substance P, trout; substance P antagonist; substance P-Gly-Lys-Arg; substance P 1-4; substance P 1-6; substance P 1-7; substance P 1-9; deca-substance P (substance P 2-11); nona-substance P (substance P 3-11); substance P tetrapeptide (substance P 8-11); substance P tripeptide (substance P 9-11); substance P, free acid; substance P methyl ester; and [Tyr⁸, Nle¹¹] substance P.

Tachykinin peptides including, but not limited to, [Ala⁵, beta-Ala⁸] neurokinin A 4-10; eledoisin; locustatachykinin I (Lom-TK-I) (Locusta migratoria); locustatachykinin II (Lom-TK-II) (Locusta migratoria); neurokinin A 4-10; neurokinin A (neuromedin L, substance K); neurokinin A, cod and trout; biotinyl-neurokinin A (biotinyl-neuromedin L, biotinyl-substance K); [Tyr⁰]-neurokinin A. [Tyr⁶]-substance K; FR64349; [Lys³, Gly⁸-(R)-gamma-lactam-Leu⁹]-neurokinin A 3-10; GR83074; GR87389; GR94800; [Beta-Ala⁸]-neurokinin A 4-10; [Nle¹⁰]-neurokinin A 4-10; [Trp⁷, beta-Ala⁸]-neurokinin A 4-10; neurokinin B (neuromedin K); biotinyl-neurokinin B (biotinyl-neuromedin K); [MePhe⁷]-neurokinin B; [Pro⁷]-neurokinin B; [Tyr⁰]-neurokinin B; neuromedin B, porcine; biotinyl-neuromedin B, porcine; neuromedin B-30, porcine; neuromedin B-32, porcine; neuromedin B receptor antagonist; neuromedin C, porcine; neuromedin N, porcine; neuromedin (U-8), porcine; neuromedin (U-25), porcine; neuromedin U, rat; neuropeptide-gamma (gamma-preprotachykinin 72-92); PG-KII; phyllolitorin; [Leu⁸]-phyllolitorin (Phyllomedusa sauvagei); physalaemin; physalaemin 1-11; scyliorhinin II, amide, dogfish; senktide, selective neurokinin B receptor peptide; [Ser²]-neuromedin C; beta-preprotachykinin 69-91, human; beta-preprotachykinin 111-129, human; tachyplesin I; xenopsin; and xenopsin 25 (xenin 25), human.

Thyrotropin-releasing hormone (TRH) peptides including, but not limited to, biotinyl-thyrotropin-releasing hormone; [Glu¹]-TRH; His-Pro-diketopiperazine; [3-Me-His²]-TRH; pGlu-Gln-Pro-amide; pGlu-His; [Phe²]-TRH; prepro TRH 53-74; repro TRH 83-106; prepro-TRH 160-169 (Ps4, TRH-potentiating peptide); prepro-TRH 178-199; thyrotropin-releasing hormone (TRH); TRH, free acid; TRH-SH Pro; and TRH precursor peptide.

Toxin peptides including, but not limited to, omega-agatoxin TK; agelenin, spider, Agelena opulenta); apamin (honeybee, Apis mellifera); calcicudine (CaC) (green mamba, Dedroaspis angusticeps); calciseptine (black mamba, Dendroaspis polylepis polylepis); charybdotoxin (ChTX) (scorpion, Leiurus quinquestriatus var. hebraeus); chlorotoxin; conotoxin GI (marine snail, Conus geographus); conotoxin GS (marine snail, Conus geographus); conotoxin MI (Marine Conus magus); alpha-conotoxin EI, Conus ermineus; alpha-conotoxin SIA; alpha-conotoxin Iml; alpha-conotoxin SI (cone snail, Conus striatus); micro-conotoxin GIIIB (marine snail, Conus geographus); omega-conotoxin GVIA (marine snail, Conus geographus); omega-conotoxin MVIIA (Conus magus); omega-conotoxin MVIIC (Conus magus); omega-conotoxin SVIB (cone snail, Conus striatus); endotoxin inhibitor; geographutoxin I (GTX-1) (μ-Conotoxin GIIIA); iberiotoxin (IbTX) (scorpion, Buthus tamulus); kaliotoxin 1-37; kaliotoxin (scorpion, Androct-onus mauretanicus mauretanicus); mast cell-degranulating peptide (MCD-peptide, peptide 401); margatoxin (MgTX) (scorpion, Centruriodes Margaritatus); neurotoxin NSTX-3 (pupua new guinean spider, Nephilia maculata); PLTX-II (spider, Plectreurys tristes); scyllatoxin (leiurotoxin I); and stichodactyla toxin (ShK); diphtheria toxin; ricin A; Pseudomonas aeruginosa exotoxin A.

Immunotoxins consist in toxins covalently linked to an antibody which acts as, a homing system, specifically targeting the toxin to those cells which one wishes to eliminate by the means of an antibody (polyclonal or monoclonal) directed against a molecule, or a group of molecules, carried at the surface of the targeted cells. Toxins including, but not limited to those cited above, can be used to this effect. The invention described in this patent application may be used to deliver such immunotoxins to the colon. In some cases, the antibody may be replaced by a small molecule that similarly acts to target the toxin to a chosen group of cells.

Vasoactive intestinal peptides (VIP/PHI) including, but not limited to, VIP, human, porcine, rat, ovine; VIP-Gly-Lys-Arg-NH2; biotinyl-PHI (biotinyl-PHI-27), porcine; [Glp¹⁶] VIP 16-28, porcine; PHI (PHI-27), porcine; PHI (PHI-27), rat; PHM-27 (PHI), human; prepro VIP 81-122, human; prepro VIP/PHM 111-122; prepro VIP/PHM 156-170; biotinyl-PHM-27 (biotinyl-PHI), human; vasoactive intestinal contractor (endothelin-beta); vasoactive intestinal octacosa-peptide, chicken; vasoactive intestinal peptide, guinea pig, biotinyl-VIP, human, porcine, rat; vasoactive intestinal peptide 1-12, human, porcine, rat; vasoactive intestinal peptide 10-28, human, porcine, rat; vasoactive intestinal peptide 11-28, human, porcine, rat, ovine; vasoactive intestinal peptide (cod, Gadus morhua); vasoactive intestinal peptide 6-28; vasoactive intestinal peptide antagonist; vasoactive intestinal peptide antagonist ([Ac-Tyr¹, D-Phe²]-GHRF 1-29 amide); vasoactive intestinal peptide receptor antagonist (4-Cl-D-Phe⁶, Leu¹⁷]-VIP); and vasoactive intestinal peptide receptor binding inhibitor, L-8-K.

Vasopressin (ADH) peptides including, but not limited to, vasopressin; [Asu^{1,6}, Arg⁸]-vasopressin; vasotocin; [Asu^{1,6}, Arg⁸]-vasotocin; [Lys⁸]-vasopressin; pressinoic acid; [Arg⁸]-desamino vasopressin desglycinamide; [Arg⁸]-vasopressin (AVP), [Arg 8]-vasopressin desglycinamide; biotinyl-[Arg⁸]-vasopressin (biotinyl-AVP); [D-Arg⁸]-vasopressin; desamino-[Arg⁸]-vasopressin; desamino-[D-Arg⁸]-vasopressin (DDAVP); [deamino-[D-3-(3′-pyridyl-Ala)]-[Arg⁸]-vasopressin; [1-(beta-Mercapto-beta, beta-cyclopentamethylene propionic acid), 2-(O-methyl)tyrosine-[Arg⁸]-vasopressin, vasopressin metabolite neuropeptide [pGlu⁴, Cys⁶]; vasopressin metabolite neuropeptide [pGlu⁴, Cys⁶]; [Lys⁸]-deamino vasopressin desglycinamide; [Lys⁸]-vasopressin; [Mpr¹, Val⁴, DArg⁸]-vasopressin; [Phe², Ile³, Orn⁸]-vasopressin ([Phe², Orn⁸]-vasotocin); [Arg⁸]-vasotocin; and [d(CH2)5, Tyr(Me)², Orn⁸]-vasotocin.

Virus related peptides including, but not limited to, fluorogenic human CMV protease substrate; HCV core protein 59-68; HCV NS4A protein 18-40 (JT strain); HCV NS4A protein 21-34 (JT strain); hepatitis B virus receptor binding fragment; hepatitis B virus pre-S region 120-145; [Ala¹²⁷]-hepatitis B virus pre-S region 120-131; herpes virus inhibitor 2; HIV envelope protein fragment 254-274; HIV gag fragment 129-135; HIV substrate; P 18 peptide; peptide T; [3,5 diiodo-Tyr⁷] peptide T; R15K HIV-1 inhibitory peptide; T20; T21; V3 decapeptide P 18-110; and virus replication inhibiting peptide.

Proteins of the Wnt Family, and Fragments Thereof.

While certain analogs, fragments, and/or analog fragments of the various polypeptides have been described above, it is to be understood that other analogs, fragments, and/or analog fragments that retain all or some of the activity of the particular polypeptide, or on the contrary that act as an antagonist thereby preventing its action, may also be useful in embodiments of the present invention. Analogs may be obtained by various means, as will be understood by those skilled in the art. For example, certain amino acids may be substituted for other amino acids in a polypeptide without appreciable loss of interactive binding capacity with structures such as, for example, antigen-binding regions of antibodies or binding sites on substrate molecules. As the interactive capacity and nature of a polypeptide drug defines its biological functional activity, certain amino acid sequence substitutions can be made in the amino acid sequence and nevertheless remain a polypeptide with like properties, or on the contrary confer to this analogue antagonistic activity that interferes with or blocks the action of the natural product. Furthermore, small molecules, whether peptidomimetic or not, natural or synthetic, may be able to substitute for the proteins and peptides cited above and have similar activity by binding to their receptors. On the contrary, such small molecules may block or interfere with the activity of the proteins and peptides cited above by various mechanisms, including, but not limited to, preventing their interaction with their cognate receptors. Additionally, many of the proteins cited above act as initiators of signaling pathways. An embodiment of this invention is the use of chemical molecules (peptides, peptidomimetics, or any other natural or synthetic molecule of any chemical nature) as activators or inhibitors of these signaling pathways. Examples of this strategy are the use of inhibitors of gamma-secretase to inhibit the Notch signaling pathway, or inhibitors of the interaction between beta-catenin and Tcf transcription factors to inhibit the Wnt-beta-catenin pathway, both of which are involved in colorectal cancer.

g) Oligonucleotide Agents

The active agents can also be in the form of oligonucleotides, including oligoribonucleotides, oligodeoxyribonucleotides and derivatives thereof useful for prophylactic, palliative or therapeutic purposes, including gene therapy and the treatment of cancer, such as colon cancer.

An oligonucleotide is a polymer of a repeating unit generically known as a nucleotide. An unmodified (naturally occurring) nucleotide has three components: (1) a nitrogen-containing heterocyclic base linked by one of its nitrogen atoms to (2) a 5-pentofuranosyl sugar and (3) a phosphate esterified to one of the 5′ or 3′ carbon atoms of the sugar. When incorporated into an oligonucleotide chain, the phosphate of a first nucleotide is also esterified to an adjacent sugar of a second, adjacent nucleotide via a 3′-5′ phosphate linkage. Nucleotides are nucleosides that further include a phosphate group covalently linked to the sugar portion of the nucleoside. In forming oligonucleotides, the phosphate groups covalently link adjacent nucleosides to one another to form a linear polymeric compound. The respective ends of this linear polymeric structure can be further joined to form a circular structure, however, within the context of the invention, open linear structures are generally preferred.

Oligonucleotides can include nucleotide sequences sufficient in identity and number to effect specific hybridization with a particular nucleic acid. Such oligonucleotides which specifically hybridize to a portion of the sense strand of a gene are commonly described as “antisense.” In the context of the invention, “hybridization” means hydrogen bonding, which may be Watson-Crick, Hoogsteen or reversed Hoogsteen hydrogen bonding, between complementary nucleotides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other.

The term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent intersugar (backbone) linkages as well as oligonucleotides having non-naturally-occurring portions which function similarly. They may be single or double stranded.

Generally, oligonucleotides formulated in the drug delivery systems of the invention may be from about 8 to about 100 nucleotides in length, more preferably from about 10 to about so nucleotides in length, and most preferably from about 10 about 25 nucleotides in length.

Oligonucleotides that are formulated in the drug delivery systems of the invention include antisense compounds and other bioactive oligonucleotides. A discussion of antisense oligonucleotides and some desirable modifications can be found in De Mesmaeker et al. (Acc. Chem. Res., 1995, 28, 366).

As used herein, antisense compounds include antisense oligonucleotides, antisense peptide nucleic acids (PNAs), small interfering RNAs, short hairpin RNAs, ribozymes and external guide sequences (EGSs). In antisense modulation of messenger RNA (mRNA), hybridization of an antisense compound with its mRNA target interferes with the normal role of mRNA and causes a modulation of its function in cells. The functions of mRNA to be interfered with include all vital functions such as translocation of the RNA to the site for protein translation, actual translation of protein from the RNA, splicing of the RNA to yield one or more mRNA species, turnover or degradation of the mRNA and possibly even independent catalytic activity which may be engaged in by the RNA. The overall effect of such interference with mRNA function is modulation of the expression of a protein, wherein “modulation” means either an increase (stimulation) or a decrease (inhibition) in the expression of the protein. In the context of the present invention, inhibition is the preferred form of modulation of gene expression.

Antisense compounds can exert their effect by a variety of means. One such means is the antisense-mediated direction of an endogenous nuclease, such as RNase H in eukaryotes or RNase P in prokaryotes, to the target nucleic acid (Chiang et al., J. Biol. Chem., 1991, 266, 18162; Forster et al., Science, 1990, 249, 783).

The sequences that recruit RNase P are known as External Guide Sequences, hence the abbreviation “EGS” (Guerrier-Takada et al., Proc. Natl. Acad. Sci. USA, 1997, 94, 8468). Another means involves covalently linking a synthetic moiety having nuclease activity to an oligonucleotide having an antisense sequence, rather than relying upon recruitment of an endogenous nuclease. Synthetic moieties having nuclease activity include, but are not limited to, enzymatic RNAs, lanthanide ion complexes, and the like (Haseloff et al., Nature, 1988, 334, 585; Baker et al., J. Am. Chem. Soc., 1997, 119, 8749).

As used herein, the term “antisense compound” also includes ribozymes, synthetic RNA molecules and derivatives thereof that catalyze highly specific endoribonuclease reactions (see, generally, U.S. Pat. No. 5,543,508 to Haseloff et al. and U.S. Pat. No. 5,545,729 to Goodchild et al.).

In addition, the term “antisense compound” includes RNAs (or DNAs that encode such RNAs) leading to the modulation of gene expression by the mechanism of RNA interference. Such molecules include, but are not limited to, short interfering RNAs, consisting of double stranded RNAs of less than 50 base pairs, typically 21 or 29 nucleotides in length with the addition at either of their extremities of other chemical molecules (including deoxyribonucleotides, natural or modified), as well as short hairpin RNAs (or DNA molecules including plasmids and viruses of any nature leading to their production, in vitro or in vivo) that act by RNA interference. This also includes any DNA or RNA molecule, single or double strand, that leads in cells to RNA interference.

The antisense compounds formulated in the drug delivery systems of the invention (1) can be from about 8 to about 100 nucleotides in length, more preferably from about 10 to about 30 nucleotides in length, (2) single or double stranded, (3) are targeted to a nucleic acid sequence required for the expression of a gene from a mammal, including a human, and (4), when contacted with cells expressing the target gene, modulate its expression. Due to the biological activity of the gene product encoded by the target gene, modulation of its expression has the desirable result of providing specific prophylactic, palliative and/or therapeutic effects.

It is understood in the art that the nucleobase sequence of an oligonucleotide or other antisense compound need not be 100% complementary to its target nucleic acid sequence to be specifically hybridizable. An antisense compound is specifically hybridizable to its target nucleic acid when there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under assay conditions.

Other bioactive oligonucleotides include aptamers and molecular decoys. As used herein, the term is meant to refer to any oligonucleotide (including a peptide-nucleic acid or PNA) that (1) provides a prophylactic, palliative or therapeutic effect to an animal in need thereof and (2) acts by a non-antisense mechanism, i.e., by some means other than by hybridizing to a nucleic acid.

The name aptamer has been coined by Ellington et al. (Nature, 1990, 346, 818) to refer to nucleic acid molecules that fit and therefore bind with significant specificity to non-nucleic acid ligands such as peptides, proteins and small molecules such as drugs and dyes. Because of these specific ligand binding properties, nucleic acids and oligonucleotides that may be classified as aptamers may be readily purified or isolated via affinity chromatography using columns that bear immobilized ligand. Aptamers may be nucleic acids that are relatively short to those that are as large as a few hundred nucleotides. For example, RNA aptamers that are 155 nucleotides long and that bind dyes such as Cibacron Blue and Reactive Blue 4 with good selectivity have been reported (Ellington et al., Nature, 1990, 346, 818). While RNA molecules were first referred to as aptamers, the term as used in the present invention refers to any nucleic acid or oligonucleotide that exhibits specific binding to small molecule ligands including, but not limited to, DNA, RNA, DNA derivatives and conjugates, RNA derivatives and conjugates, modified oligonucleotides, chimeric oligonucleotides, and gapmers (see, e.g. U.S. Pat. No. 5,523,3B9, to Ecker et al., issued Jun. 4, 1996 and incorporated herein by reference).

Molecular decoys are short double-stranded nucleic acids (including single-stranded nucleic acids designed to “fold back” on themselves) that mimic a site on a nucleic acid to which a factor, such as a protein, binds. Such decoys are expected to competitively inhibit the factor; that is, because the factor molecules are bound to an excess of the decoy, the concentration of factor bound to the cellular site corresponding to the decoy decreases, with resulting therapeutic, palliative or prophylactic effects. Methods of identifying and constructing decoy molecules are described in, e.g., U.S. Pat. No. 5,716,780 to Edwards et al.

Another type of bioactive oligonucleotide is an RNA-DNA hybrid molecule that can direct gene conversion of an endogenous nucleic acid (Cole-Strauss et al., Science, 1996, 273, 1386).

Preferred modified oligonucleotide backbones include, for example, phosphorothioates, chiral phosphorothioates, phosphoro-dithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5 to 5′-3′ or 2′-5′ to 5′-2′. Various salts, mixed salts and free acid forms are also included.

Any of the preceding bioactive oligonucleotides can be formulated into the drug delivery system of the invention and used for prophylactic or therapeutic purposes. The oligonucleotides can be stabilized through complexation, for example, with cationic lipids such as Lipoplexe or cationic polymers such as Polyplexe.

h) Diagnostic Agents

Medical imaging is the non-invasive or non-surgical visualization of internal organs or processes. Representative diagnostic methods include X-rays, magnetic resonance imaging (MRI), radionuclides or nuclear medicine, and ultrasound.

Radionuclides are nuclei that decay by dissipating excess energy (parent) to become stable (daughter) by energy emission in form of particulate or electromagnetic radiation. Fluoroscopy is a fluorescent screen that detects gamma or X-rays, which are imaged by a TV camera to afford real time images of organs in motion by using contrast agents, such as PCTA. CAT—Computed axial tomography—takes advantage of small differences in tissue radiographic density to create an image. The colon is often imaged using a lower GI series of a barium enema to conduct a radiographic study of the large bowel colon and rectum.

Technetium is a common radiolabel. Other radiolabeled compounds include iodine radiolabels, such as iobenguane sulfate ¹³¹I, sodium ¹²³iodine, sodium ¹³¹iodine, and indium labels, such as ¹¹¹In radiolabels, indium chloride, and indium satumomabpendetide. Imaging contrast agents include iron-containing contrast agents such as ferumoxides and dentritic gadolinium.

The present invention can be used to deliver to the colon agents that enable or facilitate the visualization of structures, lesions, cells carrying defined cell surface or intracellular molecules by any imaging technique including, but not restricted to, radiography, radio-tomography, magnetic resonance imaging (MRI), ultrasonic, positron emission tomography (PET scan), or any other form of imaging technique using radio-magnetic waves of whatever wavelength. For example, small molecules or antibodies that recognize cell-surface structures of colon cancer cells can be labeled with radionuclides such as ⁹⁹Technecium and used to detect tumor cells and metaslases of various sizes including micro-metastases.

### II. Methods for Preparing the Pectin Beads

Pectin beads can be prepared using methods known to those of skill in the art, including by mixing the active agent(s) in a pectin solution, and gelling the pectin anionic moieties with a divalent cation such as divalent zinc, for example, in the form of a zinc acetate solution.

The gellation is typically done by stirring a solution, suspension or dispersion of the active agent, in one embodiment, β-lactamase L1, and pectin, adjusting the pH of the solution if necessary, and adding this solution dropwise to a zinc acetate solution under agitation. In some embodiments, where the active agent(s) are not adversely affected by other metal ions, divalent or trivalent metal ions other than zinc can be used.

Suitable technologies for adding the pectin solution dropwise to the zinc acetate solution are known to those of skill in the art; and include the multi-nozzle system from Nisco Engineering AG and other relevant technologies to produce drops from a pectin solution.

The pectin drops undergo a gelification process, ideally during a predetermined time to obtain the best encapsulation yield and subsequent release efficiency.

The concentration of the pectin solution is advantageously from around 4 to around 10% (w/v), preferably around 4 to around 7%, the metal cation, such as zinc acetate, solution is advantageously from about 2 to about 20% (w/v), preferably from about 5 to about 15%. More preferably, the pectin solution is about 5% (w/v), the zinc acetate solution is about 12% (w/v).

The pectin beads are advantageously stirred in the metal cation, such as zinc acetate, solution, at a pH of about 6, at room temperature, under slow agitation, for at least around 12 minutes up to around 20 hours, preferably from around 20 minutes to around 2 hours.

The beads can then be recollected and rinsed in distilled water, ideally until the conductivity of the rinsing solution reaches a plateau. Rinsing is preferably done at least twice or under a continuous process to minimize the amount of residual zinc acetate recovered in the rinsing solution.

The rinsed beads can then be collected and can be subjected to a drying process using methods known to those of skill in the art, including heated incubator or fluidized bed technologies.

The beads are typically dried at a temperature of between around 20 and around 40° C. for around 30 min to around 24 hours, preferably at around 35° C. overnight. Drying is preferably performed until the weight of the beads reaches a plateau.

The diameter of the particles can be finely tuned using needles of appropriate internal diameter to form the pectin drops added to the zinc acetate solution. The beads are preferably between about 600 and 1500 μm in diameter.

When the active agent is β-lactamase L1, the encapsulation yields are typically between 50 and 100%, measured in terms of enzymatic activity.

### III. Formation of Drug Delivery Systems Including Pectin Beads

The pectin beads can be collected, and combined with appropriate excipients and formulated into a variety of oral drug delivery systems. For example, the beads can be combined with a solid excipient, and tableted, or included in a capsule.

The pectin beads can also be combined with liquid/gel excipients which do not degrade the pectin beads, and the mixture/dispersion can be incorporated into a capsule, such as a gel-cap.

The tablets or capsules can be coated, if desired, with a suitable enteric coating so as to assist in passing through the stomach without degradation. The pH in the stomach is of the order of 1 to 3 but it increases in the small intestine and the colon to attain values close to 7 (Hovgaard L. et al. (1996) Current Applications of Polysaccarides in Colon Targeting, Critical Reviews in Therapeutic Drug Carrier Systems, 13, 185). The drug delivery systems, in the form of tablets, gelatin capsules, spheroids and the like, can reach the colon, without being exposed to these variations in pH, by coating them with a pH-dependent polymer, insoluble in acidic pH but soluble in neutral or alkaline pH (Kinget et al. op. cit.). The polymers most currently used for this purpose are derivatives of methacrylic acid, Eudragit® L and S (Ashford M. et al. (1993), An in vivo investigation of the suitability of pH-dependent polymers for colonic targeting, International Journal of Pharmaceutics. 95, 193 and 95, 241; and David A. et al. (1997) Acrylic polymers for colon-specific drug delivery, S.T.P. Pharma Sciences, 7, 546), and, more recently, Eudragit®FS.

The drug delivery systems are administered in an effective amount suitable to provide the adequate degree of treatment or prevention of the disorders for which the compounds are administered. The efficient amounts of these compounds are typically below the threshold concentration required to elicit any appreciable side effects. The compounds can be administered in a therapeutic window in which some the disorders are treated and certain side effects are avoided. Ideally, the effective dose of the compounds described herein is sufficient to provide the desired effects in the colon but is insufficient (i.e., is not at a high enough level) to provide undesirable side effects elsewhere in the body.

Most preferably, effective doses are at very low concentrations, where maximal effects are observed to occur, with minimal side effects, and this is optimized by targeted colonic delivery of the active agents. The foregoing effective doses typically represent that amount administered as a single dose, or as one or more doses administered over a 24-hour period.

### IV. Methods of Treatment Using the Drug Delivery Systems Described Herein

The drug delivery systems described herein can be used to treat those types of conditions and disorders for which colonic delivery is appropriate. In one embodiment, the disorders are those that result from exposure of the colon to antibiotics, such as diarrhea, modification of the commensal flora and the development of bacterial resistance to antibiotics. In this embodiment, the drug delivery systems contain agents which inactivate antibiotics, and the active principles can be administered in a therapeutically effective dosage to a patient who has been, is being, or will be treated with one or several antibiotics.

In another embodiment, the drug delivery systems are administered to a patient who suffers from colon cancer. In this embodiment, the drug delivery systems include one or more antitumor agents, and the systems are administered in a therapeutically effective dosage to a patient who is suffering from colon cancer. Alternatively, the cancer can be present at another location in the body, and the drug delivery systems can be used to by-pass the stomach and its concomitant degradation of certain antitumor agents, so as to avoid the need to use intramuscular or intravenous administration of these agents.

In another embodiment, the drug delivery systems are administered to a patient who suffers from a colonic disorder such as Chrohn's disease, ulcerative colitis, irritable bowel syndrome, diarrhea, or constipation. In this embodiment, the drug delivery systems include agents which treat or prevent these disorders, and the systems can be administered in a therapeutically effective dosage to a patient who is suffering from such a disorder.

In still another embodiment, the drug delivery systems are used to administer peptide or protein-based active agents, such as insulin, antibodies, and the like, or oligonucleotide-based therapeutics, such as antisense or RNA interference therapy, so that the agents pass through the stomach without being digested. In this embodiment, the drug delivery systems include these protein/peptide/oligonucleotide-based agents, and the systems can be administered in a therapeutically effective dosage to a patient in need of treatment with these agents, without the need to administer these agents via subcutaneous or intravenous injection.

In a further embodiment, the drug delivery systems are used to administer diagnostic agents to the colon. In this embodiment, the drug delivery systems include diagnostic agents, such as imaging contrast agents, and the systems are administered in a diagnostically effective dosage to a patient who will be subjected to a diagnostic assay for diagnosis of a colonic disorder.

The present invention will be further understood with reference to the following non-limiting examples.

### EXAMPLE 1

### Development of a Sensitive, Quantitative and Specific Assay for β-Lactamase L1

Hydrolysis of nitrocefin is a well known technique used to quantify penicillinase activity. However, the usual format is in single tubes and is not adapted for analysis of a large number of samples. This example describes the development and fit for purpose qualification of this assay in 96 wells microplate format

A stock solution of nitrocefin was obtained by dissolving nitrocefin dried powder at a concentration of 10 mM in dimethylsulfoxide (DMSO). The stock solution was stored at −20° C. and diluted 100-fold immediately prior to use in 50 mM sodium phosphate buffer (Hepes buffer) pH 7.0 containing 0.1 mg/ml bovine serum albumin (BSA). Buffer selection is described in Table 1.

20 μl containing the solution to be analyzed were added to 180 μl of diluted nitrocefin. Kinetics of nitrocefin hydrolysis were followed at 37° C. with absorbance measured at 492 nm each 30 seconds using a Multiskan Ascent (Thermo Labsystems) plate reader.

The slope (difference in absorbance/second) was calculated using Excel Adds In Cellula (Prism Technologies, Cambridge UK).

β-lactamase L1 (Eurogentec, Belgium, approx. 10 mg/mL was determined by μBCA assay) was diluted 500×, 1000×, 2000× and 4000× in each solubilization buffer and reaction was initiated by adding 20 μl of solution containing enzyme to 180 μl of buffers containing nitrocefin at 100 μM.

Activity of β-lactamase L1 was tested in 10 mM Hepes, 145 mM NaCl buffer pH 7.4. The interference of EDTA with the activity of the metallo-dependent enzyme and the need for a carrier protein (Bovine Serum Albumin, abbreviated as BSA) were tested. As illustrated in Table 1. EDTA (which can be used to solubilize beads in vitro to assay their contents) should be avoided. The inclusion of BSA or other carrier proteins is beneficial.

TABLE 1
Selection of buffer drug delivery system for
β-lactamase L1 activity quantification

| Buffer | Slope | Yield |
|---|---|---|
| 10 mM Hepes, 145 mM NaCl pH 7.4 | 0.142 | 100% |
| 10 mM Hepes, 145 mM NaCl, 1% EDTA pH 7.4 | 0.026 | 18.8% |
| 10 mM Hepes, 145 mM NaCl, 0.1 mg/ml BSA pH 7.4 | 0.167 | 118.2% |
| 10 mM Hepes, 145 mM NaCl, 0.1 mg/ml BSA, 1% | 0.084 | 59.0% |
| EDTA pH 7.4 | | |

As illustrated in Table 1, EDTA interferes with the enzymatic activity assay, and BSA enhances the recovery of enzymatic activity.

### EXAMPLE 2

### Instability of β-Lactamase L1 in Original Pectin Mix and Effect of Metallic Counter-Ion

0.3 ml of β-lactamase L1 (Eurogentec, Belgium, approx. 10 mg/mL as determined by μBCA assay) was mixed to 10 g of a 6% pectin solution (Low methoxylated amidated pectin (Unipectine), Texturant Systems, cat#OG175C) made in water; the pH of the pectin solution was not adjusted.

The pectin/β-lactamase L1 mixture was added drop-wise over a period of 2 minutes using a peristaltic pump and a needle of 0.8 mm inner diameter to a beaker containing 40 ml of calcium chloride (6%) under agitation (200 rpm) at room temperature.

After further incubation to allow equilibration between free and bound calcium ions, beads were recovered by filtration and washed 3 times in 200 ml of purified water to eliminate excess of free calcium. At this stage, beads are referred to as “gelled beads”.

Beads were dried 2 hours at 37° C. in an oven, yielding dried beads.

2×5 droplets and 2×15 droplets were sampled at the exit of the needle to measure the initial β-lactamase L1 activity. Protein-free beads were also prepared as negative controls.

The β-lactamase L1 enzymatic activity (nitrocefin hydrolysis) was quantified with and without Zn ions (0.1 mM ZnCl₂) as described in example 1.

As illustrated in Table 2, no enzymatic activity was found in the β-lactamase L1/pectin mix while significant activity was recovered in the beads assayed in buffer containing Zn⁺².

TABLE 2
Inactivation of β-Lactamase L1 in non pH-adjusted pectin solution

| Slope/min | Without ZnAc | With ZnAc |
|---|---|---|
| Before mix with pectin | 0.105 | 0.103 |
| | (100.0%) | (100.0%) |
| β-Lactamase/pectin mix | 0.000 | 0.000 |
| | (0.0%) | (0.0%) |
| Gelled milli particles | 0.0078 | 0.042 |
| | (7.4%) | (40.7%) |

### EXAMPLE 3

### Optimization of Metallic Ion Used to Gel the Pectin, and the Effect of pH of the Pectin Solution

In order to determine the effects of the pectin solution parameters and zinc ions, an experiment comparing four formulations was performed. The design was built according to factorial design, Design Expert 6.0.10, Stat-Ease, Minneapolis. Two parameters were tested:

(a) pH of the pectin solution: 4.0 and 7.0

(b) cation in the gelification bath: Ca²⁺ (CaCl₂) or Zn²⁺ (Zinc acetate abbreviated ZnAc)

Beads were prepared as described in Example 2. However, the concentration of the pectin solution was decreased from 6% to 4% due to the decrease in solubility of pectin with increased pH.

The encapsulation yield was measured by assaying the enzymatic activity of β-Lactamase L1 as described in Example 1.

5 beads were solubilized in 20 ml of 10 mM Hepes, 145 mM NaCl, 0.1 mg/ml BSA at a pH of 7.4, in the presence or absence 1% pectinase (Pectinases from Aspergillus Aculeatus, Pectinex SP-1. Ultra-(SIGMA, France) overnight at 4° C.

The positive control was prepared by diluting the same amount of β-lactamase L1 as should be contained in 5 beads in 20 ml of 10 mM Hepes, 145 mM NaCl, 0.1 mg/ml BSA pH 7.4. As illustrated in Table 3, β-lactamase L1 was inactivated irrespective of the cation used for pectin gelification when the pectin solution was at pH 4.0 (4.3% residual activity in calcium and 3.8% in zinc), whereas nearly full activity was retained after buffering the pectin solution to pH 7.0 (86.7% in calcium and 64.0% in zinc):

TABLE 3
Effect of cation used for gelification and pH of pectin
on stability (recovery of β-Lactamase activity)

| Sample | CaCl₂, pH 4 | CaCl₂, pH 7 | ZuAc, pH 4 | ZnAc, pH 7 |
|---|---|---|---|---|
| Before mix | 0.102 | 0.090 | 0.108 | 0.090 |
| | (100%) | (100%) | (100%) | (100%) |
| Gelled beads | 0.004 | 0.072 | 0.004 | 0.072 |
| | (4.3%) | (80.0%) | (3.8%) | (80.0%) |
| Dried beads | 0.003 | 0.078 | 0.037 | 0.058 |
| | (31.7%) | (86.7%) | (35.0%) | (64.0%) |

### EXAMPLE 4

### Determination of Critical Parameters to Formulate β-Lactamase L1 for Colon-Specific Delivery and Optimization of These Parameters

Five parameters were tested:

(a) Concentration of the pectin solution (Low methoxylated amidated pectin (Unipectine), Texturant Systems, cat#OG175C): 4% and 5% (w/v)

(b) Cation for gelification: Ca²⁺ or Zn²+

(c) Secondary coating of the gelled beads with polyethyleneimine (PEI) solution (PEI, High molecular weight, water-free (SIGMA-ALDRICH, France))

(d) pH of the PEI solution: 7 and 11 (original non pH-adjusted solution).

(e) Solubilization of the beads to assay the encapsulated enzymatic activity with and without 1% pectinase.

Table 4 summarizes the experimental design

TABLE 4
Experimental design for the optimization of critical
parameters involved in β-Lactamase L1 formulation

| | | | | D: | |
|---|---|---|---|---|---|
| | A: | B: | C: | pH of | E: |
| Run | Pectin (%) | Ion | PEI Coating | PEI | Pectinase |
| 1 | 5 | Zn²⁺ | Yes | 11 | Yes |
| 2 | 4 | Zn²⁺ | Yes | 7 | Yes |
| 3 | 5 | Ca²⁺ | No | | Yes |
| 4 | 4 | Ca²⁺ | Yes | 7 | No |
| 5 | 5 | Ca²⁺ | Yes | 7 | Yes |
| 6 | 4 | Ca²⁺ | No | | Yes |
| 7 | 4 | Zn²⁺ | Yes | 11 | No |
| 8 | 5 | Ca²⁺ | Yes | 11 | No |
| 9 | 4 | Ca²⁺ | Yes | 11 | Yes |
| 10 | 4 | Zn²⁺ | No | | No |
| 11 | 5 | Zn²⁺ | No | | Yes |
| 12 | 4 | Zn²⁺ | No | | Yes |
| 13 | 5 | Zn²⁺ | Yes | 7 | No |
| 14 | 5 | Ca²⁺ | No | | No |
| 15 | 4 | Ca²⁺ | No | | No |
| 16 | 5 | Zn²⁺ | No | | No |

These 16 experiments were performed in duplicate (32 results).

Run 13 replicated (34 results).

The pH of the 4% and 5% pectin solutions were adjusted to 7.0. However, it was determined that the pH of the 5% pectin solution was unstable and decreased to pH 5.4 by the end of the experiments. A 5% pectin solution was therefore also adjusted to pH 8.5 for comparison.

Finally, the 48 results were analyzed using Factorial Design.

Beads were prepared as described in example 2 except that the gelification time in the cation bath was reduced from 20 min to 10 min to allow a smart timing of the experiments.

Samples (5 beads) were solibilized overnight at 4° C. in 20 ml of 10 mM Hepes, 145 mM NaCl, 0.1 mg/ml BSA pH 7.4 with and without 1% pectinase before measuring enzymatic activity (nitrocefin hydrolysis as described in example 1).

Tale 5 summarizes the experimental results obtained.

TABLE 5
Full results of Experimental design for optimizing critical
parameters involved in β-Lactamase L1 formulation

| | % | pH | | | pH of | | |
|---|---|---|---|---|---|---|---|
| Run | pectin | pectin | ion | PEI | PEI | pectinase | yield |
| 1 | 5 | 5.4 | Zn²⁺ | yes | 11 | yes | 1.201 |
| 17 | 5 | 5.4 | Zn²⁺ | yes | 11 | yes | 1.13 |
| 3b | 5 | 5.4 | Zn²⁺ | yes | 11 | yes | 1.39 |
| 11 | 5 | 5.4 | Zn²⁺ | no | | yes | 1.272 |
| 27 | 5 | 5.4 | Zn²⁺ | no | | yes | 1.36 |
| 2b | 5 | 5.4 | zn²⁺ | no | | yes | 1.044 |
| 1b | 5 | 5.4 | Zn²⁺ | yes | 7 | yes | 1.045 |
| 13 | 5 | 5.4 | Zn²⁺ | yes | 7 | no | 0.687 |
| 29 | 5 | 5.4 | Zn²⁺ | yes | 7 | no | 0.72 |
| 33 | 5 | 5.4 | Zn²⁺ | yes | 7 | no | 0.661 |
| 34 | 5 | 5.4 | Zn²⁺ | yes | 7 | no | 0.691 |
| 16 | 5 | 5.4 | Zn²⁺ | no | | no | 0.762 |
| 32 | 5 | 5.4 | Zn²⁺ | no | | no | 0.788 |
| 45 | 5 | 8.5 | Zn²⁺ | no | | yes | 0.951 |
| 38 | 5 | 8.5 | Zn²⁺ | no | | yes | 0.818 |
| 41 | 5 | 8.5 | Zn²⁺ | no | | no | 0.245 |
| 48 | 5 | 8.5 | Zn²⁺ | no | | no | 0.363 |
| 46 | 5 | 8.5 | Zn²⁺ | yes | 7 | no | 0.815 |
| 39 | 5 | 8.5 | Zn²⁺ | yes | 7 | no | 0.826 |
| 2 | 4 | 7 | Zn²⁻ | yes | 7 | yes | 1.01 |
| 18 | 4 | 7 | Zn²⁺ | yes | 7 | yes | 1.162 |
| 12 | 4 | 7 | Zn²⁺ | no | | yes | 1.165 |
| 28 | 4 | 7 | Zn²⁺ | no | | yes | 1.148 |
| 7 | 4 | 7 | Zn²⁺ | yes | 11 | no | 0.727 |
| 23 | 4 | 7 | Zn²⁺ | yes | 11 | no | 0.679 |
| 10 | 4 | 7 | Zn²⁺ | no | | no | 0.674 |
| 26 | 4 | 7 | Zn²⁺ | no | | no | 0.659 |
| 3 | 5 | 5.4 | Ca²⁺ | yes | 7 | yes | 0.094 |
| 5 | 5 | 5.4 | Ca²⁺ | yes | 7 | yes | 0.031 |
| 19 | 5 | 5.4 | Ca²⁺ | yes | 7 | yes | 0.108 |
| 21 | 5 | 5.4 | Ca²⁺ | yes | 7 | yes | 0.039 |
| 8 | 5 | 5.4 | Ca²⁺ | yes | 11 | no | 0.047 |
| 24 | 5 | 5.4 | Ca²⁺ | yes | 11 | no | 0.066 |
| 14 | 5 | 5.4 | Ca²⁺ | no | | no | 0.488 |
| 30 | 5 | 5.4 | Ca²⁺ | no | | no | 0.512 |
| 35 | 5 | 8.5 | Ca²⁺ | yes | 7 | yes | 0.35 |
| 36 | 5 | 8.5 | Ca²⁺ | yes | 7 | yes | 0.379 |
| 42 | 5 | 8.5 | Ca²⁺ | yes | 7 | yes | 0.363 |
| 43 | 5 | 8.5 | Ca²⁺ | yes | 7 | yes | 0.394 |
| 4b | 5 | 8.5 | Ca²⁺ | yes | 7 | yes | 0.53 |
| 7b | 5 | 8.5 | Ca²⁺ | yes | 7 | no | 0.704 |
| 37 | 5 | 8.5 | Ca²⁺ | yes | 11 | no | 0.029 |
| 44 | 5 | 8.5 | Ca²⁺ | yes | 11 | no | 0.029 |
| 9b | 5 | 8.5 | Ca²⁺ | yes | 11 | no | 0.737 |
| 40 | 5 | 8.5 | Ca²⁺ | | | no | 0.322 |
| 47 | 5 | 8.5 | Ca²⁺ | | | no | 0.656 |
| 6b | 5 | 8.5 | Ca²⁺ | yes | 11 | yes | 0.517 |
| 5b | 5 | 8.5 | Ca²⁺ | no | | yes | 0.656 |
| 8b | 5 | 8.5 | Ca²⁺ | no | | no | 0.967 |

Simple mono-variate statistical analysis (decreasing yield of encapsulation sorting) highlighted that an optimal formulation of β-lactamase L1 was obtained using the following parameters:

(a) A pectin concentration of 5% (maximum solubility at pH 5.4)

(b) A pectin solution neutralized to a pH of at least 5.4

(c) A zinc ion should be used

(d) A secondary coating may be further evaluated with other type of polymers

(e) A pectinase should be used to quantify formulated β-lactamase L1.

### EXAMPLE 5

### Improvement of Stability of the Beads Comprising β-Lactamase L1 in Simulated Intestinal mediul (SIM) by Increased Zinc Ion Concentration and Duration of Drying

Beads containing β-lactamase L1 were prepared as described in example 4. Increasing zinc acetate concentrations (6, 8, 10 and 12%) were tested. Further coating with or without PEI were compared.

Drying of beads was also increased from 2 hours to overnight.

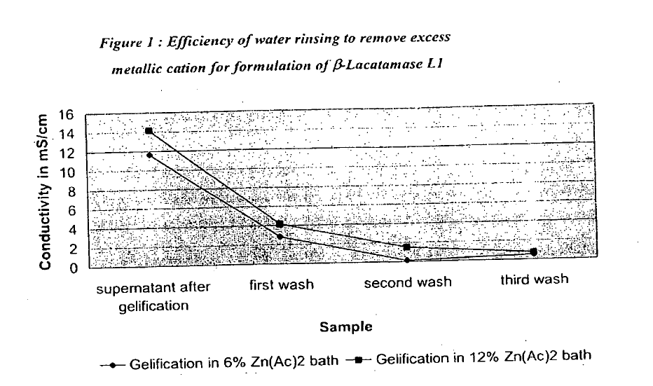

Efficiency of washing to remove excess metallic ion used for gelification was also monitored by measuring the conductivity of the water rinsing solution. As illustrated in FIG. 1, efficient washing was obtained after washing the beads in three water washes.

As illustrated in Table 6, the higher concentration of zinc acetate increased stability in SIM (Simulated Intestinal Medium, US Pharmacopeia 26) of the beads containing β-lactamase L1 while PEI secondary coating decreased their stability.

TABLE 6
Effect of Zinc acetate concentration and PEI secondary coating
on stability of beads containing β-Lactamase L1 in SIM

SIM

| Run# | % Zn | PEI | 1 h | 2 h | 3 h | 4 h | 5 h |
|---|---|---|---|---|---|---|---|
| 8 | 10% | N | + | + | + | + | + |
| 3 | 12% | Y | + | + | + | + | + |
| 4 | 12% | N | + | + | + | + | + |
| 2 | 8% | N | + | + | + | + | + |
| 5 | 6% | Y | − | − | − | − | − |
| 1 | 8% | Y | + | + | − | − | − |
| 7 | 10% | Y | + | − | − | − | − |

+: stable beads
−: dissolved beads
Y: with PEI secondary coating
N: without PEI secondary coating

### EXAMPLE 6

### Effect Of Zinc Concentration and Drying Time on the Stability of Beads in Simulated Intestinal Media (SIM)

Beads containing β-lactamase L1 were prepared as previously described, and gelled with 6 or 12% Zinc acetate solutions (see Example 5).

The effect of drying time was also tested by drying beads for 2, 4 and 16 h at 35° C. (temperature preferred to 37° C. for industrialization purposes). Only beads gelled in the 12% zinc solution and dried for more than 4 h were stable in SIM after 5 h incubation at 37° C.

TABLE 7
Stability of beads in Simulated Intestinal Medium for
5 h at 37° C. The numbers represent the number
of beads still apparently intact in solution.

| | Incubation at | 2 h | 4 h | |
|---|---|---|---|---|
| | 37° C. (h) | drying | drying | Overnight |
| milli-particles | 1 | 5 | 0 | 0 |
| in 6% Zn | 2 | 1 | 0 | 0 |
| | 3 | 1 | 0 | 0 |
| | 4 | 1 | 0 | 0 |
| | 5 | 1 | 0 | 0 |
| milli-particles | 1 | 5 | 5 | 5 |
| in 12% Zn | 2 | 5 | 5 | 5 |
| | 3 | 5 | 5 | 5 |
| | 4 | 4 | 5 | 5 |
| | 5 | 3 | 4 | 5 |

After washing and further incubation in Simulated colonic medium (SCM): 10 mM Hepes, 145 mM NaCl (stock solution). 1% pectinase, 0.1 mg/ml BSA were added just before use; pH was adjusted to pH 6.0 with NaOH 1 M, 63% of the initial β-lactamase activity (nitrocefin hydrolysis) were recovered.

### EXAMPLE 7

### Effect of Gelification Time, Rinsing Process, and Drying Time on Recovery of β-Lactamase L1 Activity

Different batches of beads were prepared using a multi-nozzle system from Nisco Engeneering AG. The beads underwent various gelification times, rinsing process and time and drying process type and time.

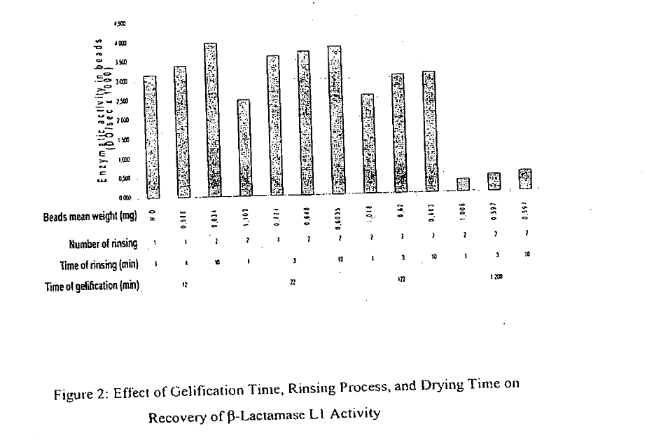

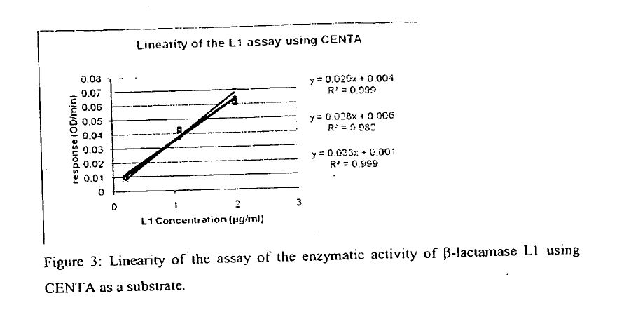

It appears clearly that the best encapsulation efficiency and enzyme activity are obtained when gelification time is less than 20 hours and when rinsing is performed such as to eliminate residual Zinc acetate from the beads. Results are presented in FIG. 2.

### EXAMPLE 8

### Development of a Sensitive, Quantitative and Specific Assay for β-Lactamase L1

Hydrolysis of CENTA is a well known technique used to quantify β-lactamase activity. However, the usual format is in single tubes and is not adapted for analysis of a large number of samples. This example describes the development and fit for purpose qualification of this assay in 96 wells microplate format

A stock solution of CENTA was obtained by solubilization of the CENTA dried powder at a concentration of 25 mM in water; it was stored in 25 μl aliquots at −20° C. The assay mix was done by diluting 22 μl of CENTA stock solution in the following assay buffer: 10 ml 30 mM Hepes buffer pH 7.5 containing 50 μm ZnCl₂, hence yielding a CENTA concentration of 110 μM. For the assay, 20 μl containing the enzyme to be assayed were added to 180 μl of assay mix, hence using a final concentration of 100 μM CENTA In the assay. Kinetics of CENTA hydrolysis were followed at 37° C. with a measure of absorbance at 405 nm each 9 seconds using a Multiskan Ascent (Thermo Electron Corporation) plate reader. The slope (difference in absorbance/second) was calculated using Ascent Software for Multiskan Ascent version 2.6.

β-lactamase L1 (Eurogentec, Belgium, approx. 10 mg/mL as determined by μBCA assay) was diluted to 0.2, 0.5, 1.0 and 2.0 μg/ml in assay buffer and the reaction was initiated by addition of 20 μl of enzyme-containing solution to 180 μl of assay mix. As shown in Figure below, the assay was linear in 3 independent assays with respect to enzyme concentration in that range. Standard deviation was less than 10%.

### EXAMPLE 9

### Release of β-Lactamase L1 from Uncoated Beads, and Eudragit-Coated Beads with or without HPMC Pre-Coating

A batch of pectin beads containing β-lactamase L1 was manufactured under the following conditions: beads were formed by adding dropwise through a 0.5 mm internal diameter needle a solution of 5% pectin containing 300 mg/l purified recombinant β-lactamase L1 (Eurogentec, Belgium) to a 12% bath of Zn acetate, 2H₂O. Beads were gelified for 90 min in the Zn acetate bath, collected, washed with water until the water conductivity had reached a stable plateau, signifying that rinsing is optimal and finally dried at 35° C. under vacuum. Dried beads obtained were 0.8-1.25 mm diameter, weighed on average 0.6 mg and contain approx 5 to 6 μg β-lactamase L1 per mg of beads. They were either left uncoated, or coated using a Glatt GPC 1.1 with Top spray according to the following formulas shown in Table 9.

TABLE 9

| | Amount | Amount | Amount | Amount | Amount | Amount |
|---|---|---|---|---|---|---|
| | (g) | (g) | (g) | (g) | (g) | (g) |
| Raw materials | Batch 83 | Batch 100 | Batch 82 | Batch 99 | Batch 81 | Batch 97 |
| Eudragit L30D-55 | 1600.0 | 149.5 | 300.0 | 31.9 | | |
| Eudragit NE 30 D | | | 700.0 | 74.4 | | |
| Eudragit FS30D | | | | | 800.0 | 85.0 |
| GMS (Glycerol | 24.0 | 2.2 | 15.0 | 1.6 | 12.0 | 1.3 |
| monostearate) | | | | | | |
| Sodium Hydroxide | 28.8 | 2.7 | 30.4 | 1.9 | | 1.5 |
| Tween 80 | 48.0 | 2.2 | 18.0 | 1.6 | 14.4 | 1.3 |
| (polysorbate) | | | | | | |
| 33% Aqueous | | | | | | |
| solution | | | | | | |
| Triethyl Citrate | 1107.2 | 94.5 | 4.50 | 67.2 | 10.0 | 25.2 |
| Water | 1600.0 | 149.5 | 565.7 | 1600.0 | 505.6 | 85.0 |
| Pre-coating with 5% | NO | YES | NO | YES | NO | YES |
| HPMC | | | | | | |

Pre-coating of beads was performed with HPMC using same material as for the coating with Eudragit.

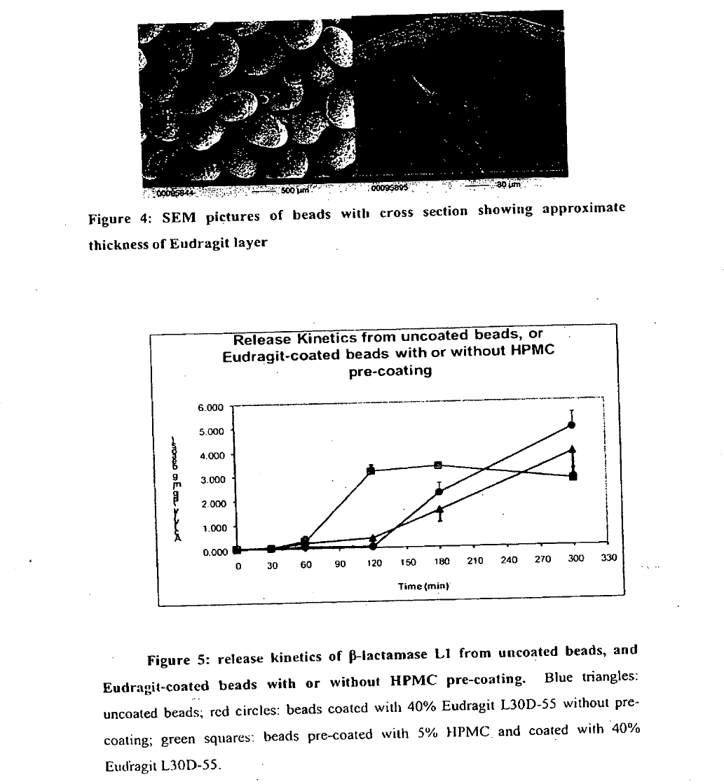

Scanning electron micrographs (SEMs) of Eudragit-coated beads are shown in FIG. 4. A cross-section shows the relative thickness of the Eudragit coating.

In order to assess the release of β-lactamase L1, coated and uncoated beads were incubated under gentle mixing at 37° C. in 50 mM Hepes buffer pH 7.4 containing 0.1 M NaCl and 100 PG/ml pectinases from Aspergillus aculeatus (Sigma Aldrich). Medium was withdrawn at various times and assayed for β-lactamase activity using the nitrocephin assay described in Example 1.

Release kinetics were measured using the coated and uncoated beads, and the results are shown in FIG. 5.

### EXAMPLE 10

### Efficiency of Released L1 to Hydrolyze Antibiotics In Vitro

In order to assess whether coated beads would actually be able to hydrolyze antibiotics when they reach the colon, they were successively incubated For 1 h in simulated gastric medium (0.1N HCl), 3 h at 37° C. in simulated intestinal medium (50 mM Na/K phosphate buffer pH 6.8 containing 0.1 M NaCl) and finally for the indicated amounts of time in simulated colonic medium (50 mM Hepes buffer pH 7.4, 0.1 M NaCl) containing 100 PG/ml pectinases from Aspergillus aculeatus (Sigma Aldrich) and 2 mg/ml amoxicillin. Medium was withdrawn at various times and the amount of residual amoxicillin was measure by HPLC and UV absorption. The procedure was performed using a Bio-Diss III apparatus (Varian). Uncoated beads were only incubated in the simulated colonic medium with pectinases and amoxicillin.

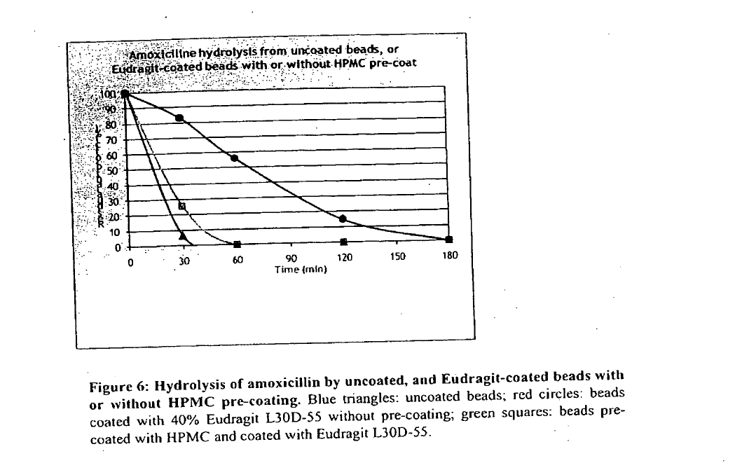

The results are shown in FIG. 6.

### EXAMPLE 11

### Effect of β-Lactamase L1 Containing Beads on the Emergence of Bacterial Resistance in Piglets Treated with Amoxicillin

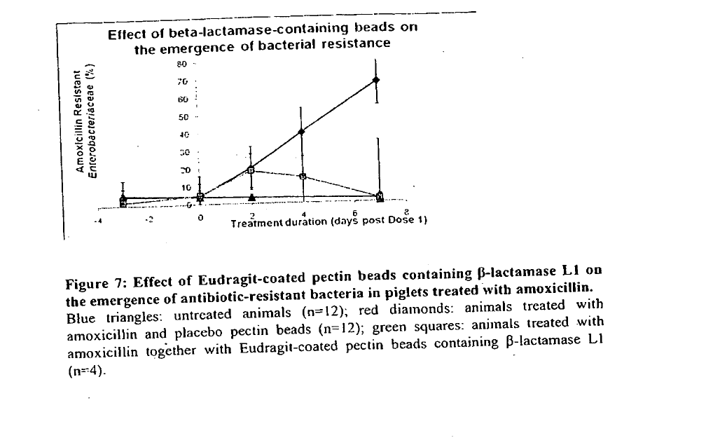

6-7 week old piglets were either untreated, or orally treated with 20 mg/kg amoxicillin per day for 7 days. Half of the treated animals received, together with the daily dose of antibiotics, a gelatin capsule filled with 320 mg pectin beads containing β-lactamase L1, pre-coated with 5% HPMC and coated with 40% Eudragit 1.30D-55 (batch 100); the other half received similarly coated placebo pectin beads. Feces were collected 3 days before the onset of treatment, and each day during 7 days of treatment and analyzed for their content of total and amoxicillin-resistant enterobacteria on MacConkey agar plates containing 0 or 100 μg/ml amoxicillin. As shown in FIG. 7, the feces of untreated animals contained a minimal proportion of amoxicillin-resistant bacteria (<5%), whereas this proportion rapidly increased in animals treated with amoxicillin, reaching a value between 50 and 80% after 7 days. In contrast, animals receiving β-lactamase containing beads together with amoxicillin only exhibited a transient and limited increase in antibiotic-resistant bacteria. This experiment shows that the co-administration of Eudragit-coated pectin beads containing β-lactamase L1 protected piglets against the emergence of antibiotic resistant bacteria induced by the treatment of animals with amoxicillin.

All patents and publications disclosed herein are incorporated by reference in their entirety. Modifications and variations of the present invention will be obvious to those skilled in the art from the foregoing detailed description of the invention.

1. A drug delivery system for oral administration and colonic delivery of a prophylactic, therapeutic or diagnostic agent, comprising a pectin bead containing a prophylactic, therapeutic or diagnostic agent, wherein the pectin is crosslinked with a metal cation and the bead is coated with a Eudragit® polymer.

2. The drug delivery system of claim 1, wherein the agent is an anti-cancer drug.

3. The drug delivery system of claim 1, wherein the agent is an anti-inflammatory.

4. The drug delivery system of claim 1, wherein the agent is a protein or peptide.

5. The drug delivery system of claim 1, wherein the agent is or comprises a nucleic acid.

6. The drug delivery system of claim 1, wherein the agent is a virus, bacteria, or fungus. structures of natural, recombinant or synthetic origin, including, but not limited to, viruses (including DNA and RNA viruses, targeting animal cells, plant cells, or bacteria);

7. The drug delivery system of claim 1, wherein the agent is a diagnostic agent.

8. The drug delivery system of claim 1, wherein the agent is an immuno-modifying agent.

9. The drug delivery system of claim 1, wherein the agent blocks or modulates the activity of receptors in the colon.

10. The drug delivery system of claim 1, wherein the agent inactivates other therapeutic agents which might modulate the activity of receptors in the colon.

11. The drug delivery system of claim 1, wherein the agent is capable of inactivating an antibiotic in the colon.

12. The drug delivery system of claim 1, wherein the metal cation is a zinc cation.

13. Oral drug delivery systems for colonic release of active ingredients, comprising.
a) an active agent capable of treating disorders or the colon, and
b) a drug delivery system comprising pectin beads, where the pectin is crosslinked with zinc ions, and the beads are coated with a Eudragit® polymer.

14. The drug delivery system of claim 13, wherein the disorder is Crohn's disease or ulcerative colitis, and the active agent is selected from the group consisting of minosalicylates, drugs that contain 5-aminosalicyclic acid (5-ASA), corticosteroids, immunomodulators, cyclosporine A, TNF alpha, thiazolidinediones and glitazones.

15. The drug delivery system of claim 14, wherein the immunomodulators are selected from the group consisting of cytokines, lymphokines and interleukins.

16. A method of treating Chrohn's disease or ulcerative colitis, comprising administering an effective amount of the drug delivery system of claim 14 to a patient in need of treatment thereof.

17. The drug delivery system of claim 13, wherein the disorder is colon cancer, and the active agent is selected from the group consisting of anti-proliferative agents, agents for DNA modification or repair, DNA synthesis inhibitors, DNA/RNA transcription regulators, RNA processing inhibitors, agents that affect protein expression, synthesis and stability, agents that affect protein localization or their ability to exert their physiological action, agents that interfere with protein-protein or protein-nucleic acid interactions, agents that act by RNA interference, receptor binding molecules of any chemical nature (including small molecules and antibodies), targeted toxins, enzyme activators, enzyme inhibitors, gene regulators, HSP-90 inhibitors, molecules interfering with microtubules or other cytoskeletal components or cell adhesion and motility, agents for phototherapy, and therapy adjuncts.

18. A method of treating colon cancer, comprising administering an effective amount of the drug delivery system of claim 17 to a patient in need of treatment thereof.

19. The drug delivery system of claim 13, wherein the disorder is irritable bowel syndrome or constipation, and the active agent is selected from the group consisting of stimulant laxatives, osmotic laxatives, stool softeners, bulking agents, Zelnorm (tegaserod), and anticholinergic medications.

20. A method of treating irritable bowel syndrome or constipation, comprising administering an effective amount of the drug delivery system of claim 19 to a patient in need of treatment thereof.

21. The drug delivery system of claim 13, wherein the system is used as a diagnostic agent, and the encapsulated agent is a diagnostic agent.

22. The drug delivery system of claim 21, wherein the diagnostic agent is selected from the group consisting of radiolabeled compounds, radioopaque compounds, and gases.

23. A method of diagnosing a disorder in the colon, comprising:
a) administering an effective amount of the drug delivery system of claim 21 to a patient in need of diagnosis thereof, and
b) detecting the diagnostic agent.
